(19) United States
(12) Patent Application Publication
(10) Pub. No.: US 2024/0074680 A1
(43) Pub. Date: Mar. 7, 2024

(54) DETECTING AND MONITORING OXYGEN-RELATED EVENTS IN HEMODIALYSIS PATIENTS

(71) Applicant: Fresenius Medical Care Holdings, Inc., Waltham, MA (US)

(72) Inventors: Alhaji Cherif, Queens, NY (US); Peter Kotanko, New York, NY (US); Paulo Paneque Galuzio, Curitiba (BR); Hanjie Zhang, Rutherford, NJ (US)

(21) Appl. No.: 18/498,844

(22) Filed: Oct. 31, 2023

Related U.S. Application Data
(63) Continuation of application No. PCT/US21/47740, filed on Aug. 26, 2021.
(60) Provisional application No. 63/192,622, filed on May 25, 2021.

(51) Int. Cl. A61B 5/145; A61B 5/00; A61M 1/16

(52) U.S. Cl. CPC A61B 5/14542; A61B 5/4809; A61B 5/4818; A61B 5/6866; A61B 5/746; A61M 1/1613; A61B 5/0826

(57) ABSTRACT

The present teachings include analyzing oxygen saturation levels sensed during a hemodialysis treatment for a patient to determine whether the patient has a medical condition based on hypoxemia, apnea, or the like experienced during the treatment. To this end, the present teachings may include the use of a machine-learning algorithm trained to identify a presence of a high-frequency intermittent pattern that would be formed in a plot of the oxygen saturation levels, e.g., to determine a severity of respiratory instability experienced. The present teaching may also or instead include a time-series analysis including at least one of: (i) calculating recurrence-based quantification, such as, but not limited to, recurrence rate, determinism, and laminarity; (ii) calculating the optimal recurrence threshold based on maximum variations of the system's determinism and degree of predictability; and (iii) calculating complexity-based measures such as permutation entropy. Such analyses may be used to detect, inter-alia, sleep apnea syndrome.

### CROSS-REFERENCE TO RELATED APPLICATIONS

This application is a bypass continuation that claims priority to International Patent Application No. PCT/US21/47740 filed on Aug. 26, 2021, which claims priority to U.S. Provisional Patent Application No. 63/192,622 filed on May 25, 2021, where the entire contents of each of the foregoing is hereby incorporated by reference.

### FIELD

The present disclosure generally relates to detecting intradialytic sleep apnea—and other conditions related to fluctuations in arterial oxygen saturation (SaO₂)—in hemodialysis patients and the like.

### BACKGROUND

Intermittency may refer to the irregular alternation of cyclic dynamics (e.g., between periodic and/or different forms of chaotic dynamics). In a physiological context, intermittent dynamics are sometime seen as beneficial, especially when induced therapeutically. However, in many pathophysiologic contexts, for example in the case of a patient receiving a hemodialysis treatment, intermittent dynamics may be associated with an increased risk of morbidity and mortality. Intermittent dynamics may appear in patients experiencing sleep apnea syndrome, chronic obstructive pulmonary disease, a stroke and/or a proclivity for strokes, hypopnea, hypoxemia, and the like. For example, sleep apnea syndrome (SAS) has recently emerged as an important cardiovascular risk factor in hemodialysis (HD) patients. In patients with SAS, high-frequency oscillations of arterial oxygen saturation (SaO₂) and arousal can be used to characterize repetitive respiratory cessation. These non-stationary dynamics can present intermittent behaviors. Although these repetitive patterns have been observed in SAS patients, it has generally not been studied during HD treatments. There remains a need for detecting such intermittent dynamics during dialysis, such as intradialytic SAS, hypopnea, hypoxemia, and/or other conditions that can be characterized by fluctuations in SaO₂, in HD patients, for example, to provide useful prognosticators of adverse events related to SAS and others.

### SUMMARY

The present teachings include analyzing oxygen saturation levels sensed during a hemodialysis treatment for a patient to determine whether the patient has a medical condition based on hypoxemia, apnea, or the like experienced during the treatment. To this end, the present teachings may include the use of a machine-learning algorithm trained to identify a presence of a high-frequency intermittent pattern that would be formed in a plot of the oxygen saturation levels, e.g., to determine a severity of respiratory instability experienced. The present teaching may also or instead include a time-series analysis including at least one of: (i) calculating recurrence-based quantification, such as, but not limited to, recurrence rate, determinism, and laminarity; (ii) calculating the optimal recurrence threshold based on maximum variations of the system's determinism and degree of predictability; and (iii) calculating complexity-based measures such as permutation entropy. Such analyses may be used to detect intermittent dynamics observed in sleep apnea syndrome, intermittent hypoxemia, and the like.

In an aspect, a method disclosed herein for detecting one or more oxygen-related events experienced during a hemodialysis procedure may include: sensing an attribute of blood of a patient within a portion of an extracorporeal circuit during a hemodialysis procedure, and analyzing the attribute to provide time-series data including a plurality of oxygen saturation levels for the patient; analyzing the time-series data using a machine-learning algorithm trained to identify a presence of an intermittent pattern that would be formed in a plot of the plurality of oxygen saturation levels experienced during the hemodialysis procedure; when the presence of the intermittent pattern is identified by the machine-learning algorithm, determining a severity of respiratory instability experienced during the hemodialysis procedure based at least in part on a value of one or more of the plurality of oxygen saturation levels; and determining whether the patient has one or more medical conditions based on the analysis of the time-series data by the machine-learning algorithm and the determined severity of respiratory instability experienced during the hemodialysis procedure.

Implementations may include one or more of the following features. The intermittent pattern may include a sawtooth pattern. The method may further include, when the presence of the intermittent pattern is identified by the machine-learning algorithm, transmitting a signal indicating that intermittent hypoxemia was experienced during the hemodialysis procedure. The signal may cause a notification to be sent to a computing device associated with one or more of the patient, a patient caregiver, a technician, and a medical professional. The notification may be sent during the hemodialysis procedure. The notification may include at least one of a visual, an audio, and a tactile alert provided by a component associated with the hemodialysis procedure. The component associated with the hemodialysis procedure may include one or more of a blood monitor and a dialysis machine. The notification may be based on information obtained in a current hemodialysis procedure. The notification may be based at least in part on information obtained over a plurality of previous hemodialysis procedures. The signal may trigger an intervention for the patient. An output of the machine-learning algorithm may include a lack of the presence of the intermittent pattern, and the lack of the presence of the intermittent pattern may indicate a lack of intermittent hypoxemia experienced during the hemodialysis procedure. The method may further include transmitting a signal indicating the lack of intermittent hypoxemia experienced during the hemodialysis procedure. The method may further include providing an intervention for the patient based on the analysis of the time-series data by the machine-learning algorithm and the determined severity of respiratory instability experienced during the hemodialysis procedure. The intervention may include one or more of awakening the patient from sleep, an adjustment to one or more settings associated with the hemodialysis procedure, polysomnography, oxygen supplementation, a medication, and an adjustment to existing medication for the patient. The intervention may be based on information obtained in a current hemodialysis procedure. The intervention may be based at least in part on information obtained over a plurality of previous hemodialysis procedures. The determined severity of respiratory instability may include at least one of mild and severe. The severity of respiratory instability may be determined to be severe if a predetermined number of the plurality of oxygen saturation levels are below 90 percent. The predetermined number may be a single oxygen saturation level. One or more of the medical conditions may include at least one of sleep apnea syndrome, chronic obstructive pulmonary disease, a stroke, a proclivity for strokes, and hypopnea. The machine-learning algorithm may be trained using one or more deep learning methods for time series classification. One or more of the deep learning methods may include a one-dimensional convolutional neural network (1D-CNN). Sensing the attribute may be conducted during one or more episodes of sleep for the patient. Sensing the attribute may include at least 30 consecutive arterial oxygen saturation recordings. The at least 30 consecutive arterial oxygen saturation recordings may be taken about every 10 seconds for 5 minutes. The method may further include monitoring the patient to determine whether the patient is sleeping. Monitoring the patient may include visually monitoring of the patient. The method may further include monitoring the patient to determine when the patient is awakened from sleep. Monitoring the patient may be conducted at least in part by a wearable physiological monitor. The method may further include monitoring snoring of the patient. The attribute may include hemoglobin. The attribute itself may be oxygen saturation. The method may further include sensing one or more second attributes of blood of the patient within the portion of the extracorporeal circuit during the hemodialysis procedure, the one or more second attributes including at least one of hematocrit and blood volume. The attribute may be sensed at a frequency of 0.1 Hertz (Hz).

In an aspect, a computer program product disclosed herein may include computer-executable code embodied in a non-transitory computer-readable medium that, when executing on one or more computing devices, performs the steps of sensing an attribute of blood of a patient within a portion of an extracorporeal circuit during a hemodialysis procedure, and analyzing the attribute to provide time-series data including a plurality of oxygen saturation levels for the patient; analyzing the time-series data using a machine-learning algorithm trained to identify a presence of an intermittent pattern that would be formed in a plot of the plurality of oxygen saturation levels experienced during the hemodialysis procedure; when the presence of the intermittent pattern is identified by the machine-learning algorithm, determining a severity of respiratory instability experienced during the hemodialysis procedure based at least in part on a value of one or more of the plurality of oxygen saturation levels; and determining whether the patient has one or more medical conditions based on the analysis of the time-series data by the machine-learning algorithm and the determined severity of respiratory instability experienced during the hemodialysis procedure.

In an aspect, a system disclosed herein may include: an extracorporeal circuit connected to a patient for performing a hemodialysis procedure; a dialysis machine within the extracorporeal circuit; a blood monitor within the extracorporeal circuit; and a computing resource configured to receive time-series data including a plurality of oxygen saturation levels for the patient. The computing resource may include computer-executable code embodied in a non-transitory computer-readable medium that, when executing on the computing resource, performs the steps of: analyzing the time-series data using a machine-learning algorithm trained to identify a presence of an intermittent pattern that would be formed in a plot of the plurality of oxygen saturation levels experienced during the hemodialysis procedure; when the presence of the intermittent pattern is identified by the machine-learning algorithm, determining a severity of respiratory instability experienced during the hemodialysis procedure based at least in part on a value of one or more of the plurality of oxygen saturation levels; and determining whether the patient has one or more medical conditions based on the analysis of the time-series data by the machine-learning algorithm and the determined severity of respiratory instability experienced during the hemodialysis procedure. The computing resource may be disposed remote from the blood monitor, and may communicate with the blood monitor over a data network.

In an aspect, a method disclosed herein for detecting one or more oxygen-related events experienced during a treatment employing extracorporeal circulation may include: sensing an attribute of blood of a patient within a portion of an extracorporeal circuit, and analyzing the attribute to provide time-series data including a plurality of oxygen saturation levels for the patient; analyzing the time-series data using a machine-learning algorithm trained to identify a presence of an intermittent pattern that would be formed in a plot of the plurality of oxygen saturation levels experienced during extracorporeal circulation; when the presence of the intermittent pattern is identified by the machine-learning algorithm, determining a severity of respiratory instability experienced during extracorporeal circulation based at least in part on a value of one or more of the plurality of oxygen saturation levels; and determining whether the patient has one or more medical conditions based on the analysis of the time-series data by the machine-learning algorithm and the determined severity of respiratory instability experienced during extracorporeal circulation.

The present teachings include systems and techniques for analyzing oxygen saturation levels sensed during a hemodialysis treatment for a patient to determine whether the patient has one or more medical conditions based on an oxygen-related event experienced during the hemodialysis procedure. To this end, the present teachings may include performing a time-series analysis on a plurality of oxygen saturation levels for the patient including at least one of: (i) calculating recurrence-based quantification, such as, but not limited to, recurrence rate, determinism, and laminarity; (ii) calculating the optimal recurrence threshold based on maximum variations of the system's determinism and degree of predictability; and (iii) calculating complexity-based measures such as permutation entropy. One or more of the aforementioned time series analyses may be used to detect sleep apnea syndrome, e.g., when the oxygen saturation levels are sensed during a sleep event while the patient is undergoing the hemodialysis treatment.

In an aspect, a method for detecting an oxygen-related event experienced during a hemodialysis procedure disclosed herein may include: sensing an attribute of blood of a patient within a portion of an extracorporeal circuit during a hemodialysis procedure over a first time period, and analyzing the attribute to provide a plurality of oxygen saturation levels for the patient over the first time period; analyzing the plurality of oxygen saturation levels, where the analysis at least in part includes determining whether one or more of the plurality of oxygen saturation levels is less than a predetermined threshold level by a predetermined amount, and application of one or more of a recurrence-based metric and a complexity-based metric to identify a possible medical condition of the patient; and determining whether the patient has one or more medical conditions based on an oxygen-related event experienced during the hemodialysis procedure based on the analysis of the plurality of oxygen saturation levels.

Implementations may include one or more of the following features. The oxygen-related event may include one or more of hypoxemia, apnea, hypopnea, and hypoxia. The first time period may include one or more episodes of sleep for the patient. One or more of the medical conditions may include sleep apnea syndrome. One or more of the medical conditions may include hypopnea. The method may further include conducting a time-series analysis on the plurality of oxygen saturation levels for the patient over at least the first time period. The time-series analysis may include calculating one or more recurrence-based metrics. Calculating one or more recurrence-based metrics may include at least one of: (i) calculating a probability of recurrence (recurrence rate) for one or more of the plurality of oxygen saturation levels; (ii) calculating predictability (determinism) for one or more of the plurality of oxygen saturation levels; (iii) identifying a rate of occurrence of one or more laminar states (laminarity); (iv) calculating an optimal recurrence threshold; and (v) calculating a complexity metric. Each of the time series analyses (i)-(v) may be collectively used to detect sleep apnea syndrome. In an aspect, (ii) calculating the predictability and (v) calculating the complexity metric are analyzed at an onset of a sleep period. The time-series analysis may include calculating a complexity metric including at least permutation entropy. The method may further include detecting an onset of intradialytic sleep apnea syndrome characterized by one or more of high-frequency and intermittent oscillatory patterns in oxygen saturation levels based on the time-series analysis. The method may further include monitoring the patient to determine whether the patient is sleeping. Monitoring the patient may include visually monitoring of the patient. The method may further include monitoring the patient to determine when the patient is awakened from sleep. Monitoring the patient may be conducted at least in part by a wearable physiological monitor. The method may further include monitoring snoring of the patient. The method may further include providing a notification regarding an oxygen-related event experienced during the hemodialysis procedure. The notification may be sent to a computing device associated with one or more of the patient, a technician, and a medical professional. The notification may be provided by at least one of a visual, an audio, and a tactile alert provided by a component associated with the hemodialysis procedure. The component associated with the hemodialysis procedure may include one or more of a blood monitor and a dialysis machine. The notification may be based on information obtained in a current hemodialysis procedure. The notification may be based at least in part on information obtained over a plurality of previous hemodialysis procedures. The method may further include providing an intervention for the patient. The intervention may include awakening the patient from sleep. The intervention may include an adjustment to one or more settings associated with the hemodialysis procedure. The intervention may include one or more of polysomnography and oxygen supplementation. The intervention may include a medication. The intervention may include an adjustment to existing medication for the patient. The intervention may be based on information obtained in a current hemodialysis procedure. The intervention may be based at least in part on information obtained over a plurality of previous hemodialysis procedures. The attribute may include hemoglobin. The attribute itself may be oxygen saturation. The method may further include sensing one or more second attributes of blood of the patient within the portion of the extracorporeal circuit during the hemodialysis procedure, the second attributes including at least one of hematocrit and blood volume. The attribute may be sensed at a frequency of 1 hertz (Hz). The predetermined amount may be about 3% below the predetermined threshold level.

In an aspect, a computer-program product disclosed herein may include computer-executable code embodied in a non-transitory computer-readable medium that, when executing on one or more computing devices, performs the steps of: sensing an attribute of blood of a patient within a portion of an extracorporeal circuit during a hemodialysis procedure over a first time period, and analyzing the attribute to provide a plurality of oxygen saturation levels for the patient over the first time period; analyzing the plurality of oxygen saturation levels, where the analysis at least in part includes determining whether one or more of the plurality of oxygen saturation levels is less than a predetermined threshold level by a predetermined amount, and application of one or more of a recurrence-based metric and a complexity-based metric to identify a possible medical condition of the patient; and determining whether the patient has one or more medical conditions based on an oxygen-related event experienced during the hemodialysis procedure from the analysis of the plurality of oxygen saturation levels.

In an aspect, a system disclosed herein may include an extracorporeal circuit connected to a patient for performing a hemodialysis procedure, a dialysis machine within the extracorporeal circuit, a blood monitor within the extracorporeal circuit, and a computing resource configured to receive data from the blood monitor related to a plurality of oxygen saturation levels for the patient over a first time period. The computing resource may include computer-executable code embodied in a non-transitory computer-readable medium that, when executing on the computing resource, performs the steps of: analyzing the plurality of oxygen saturation levels, where the analysis at least in part includes one or more of (i) determining whether one or more of the plurality of oxygen saturation levels is less than a predetermined threshold level by a predetermined amount, and (ii) application of one or more of a recurrence-based metric and a complexity-based metric to identify a possible medical condition of the patient; and determining whether the patient has one or more medical conditions based on an oxygen-related event experienced during the hemodialysis procedure from the analysis of the plurality of oxygen saturation levels. The computing resource may be disposed remote from the blood monitor, and may communicate with the blood monitor over a data network.

In an aspect, a method for detecting an oxygen-related event experienced during a treatment employing extracorporeal circulation disclosed herein may include: sensing an attribute of blood of a patient within a portion of an extracorporeal circuit during a treatment over a first time period, and analyzing the attribute to provide a plurality of oxygen saturation levels for the patient over the first time period; analyzing the plurality of oxygen saturation levels, where the analysis at least in part includes determining whether one or more of the plurality of oxygen saturation levels is less than a predetermined threshold level by a predetermined amount, and application of one or more of a recurrence-based metric and a complexity-based metric to identify a possible medical condition of the patient; and determining whether the patient has one or more medical conditions based on an oxygen-related event experienced during the treatment from the analysis of the plurality of oxygen saturation levels.

In an aspect, a method for detecting an oxygen-related event experienced during a hemodialysis procedure disclosed herein may include: sensing an attribute of blood of a patient within a portion of an extracorporeal circuit during a hemodialysis procedure over a time period, and analyzing the attribute to provide a plurality of oxygen saturation levels for the patient over the time period; analyzing the plurality of oxygen saturation levels, where the analysis at least in part includes application of a recurrence-based metric yielding a recurrence threshold to identify presence of the oxygen-related event; when the recurrence threshold is less than a predetermined minimum value, εₘᵢₙ, determining a lack of presence of the oxygen-related event; when the recurrence threshold is greater than or equal to a predetermined maximum value, εₘₐₓ, determining that the oxygen-related event is present; and, when the recurrence threshold is greater than or equal to εₘᵢₙ but less than εₘₐₓ, analyzing the plurality of oxygen saturation levels over the time period using a machine-learning algorithm trained to identify a presence of an intermittent pattern that would be formed in a plot of the plurality of oxygen saturation levels.

These and other features, aspects, and advantages of the present teachings will become better understood with reference to the following description, examples, and appended claims.

### BRIEF DESCRIPTION OF THE DRAWINGS

The foregoing and other objects, features and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 is a system for monitoring oxygen saturation (SaO₂) in hemodialysis patients, in accordance with a representative embodiment.

FIG. 2 is a flow chart of a method for monitoring SaO₂ in hemodialysis patients, in accordance with a representative embodiment.

FIG. 3 shows observations from an example study described herein.

FIG. 4 shows a conceptual illustrative oxygen desaturation episode close-up with desaturation event determination from the example study described herein.

FIG. 5 is a dendrogram with the clustering of patients from the example study described herein.

FIG. 6 is the time series of two representative patients in the example study described herein.

FIG. 7 shows the receiver operating characteristic (ROC) curves for two metrics that successfully diagnosed the patients in the example study described herein.

FIG. 8 shows a time series for the oxygen desaturation density (ODD) superimposed to corresponding oxygen saturation for comparison, as discussed in the example study described herein.

FIG. 9 is a scatter plot of the metrics for the different intensities of the oxygen desaturation index used in the example study described herein.

FIG. 10 shows ROC curves for different binary classifiers according to the example study described herein.

FIG. 11 is a graph demonstrating continuous and intermittent hypoxemia patterns for context.

FIG. 12 shows an example of an intermittent pattern in oxygen saturation levels detected in a hemodialysis patient.

FIG. 13 shows an example of an intermittent pattern in oxygen saturation levels detected in a hemodialysis patient.

FIG. 14 shows an example of an intermittent pattern in oxygen saturation levels detected in a hemodialysis patient.

FIG. 15 shows an example of an intermittent pattern in oxygen saturation levels detected in a hemodialysis patient.

FIG. 16 is a flow chart of a method for detecting one or more oxygen-related events experienced during a treatment employing extracorporeal circulation, in accordance with a representative embodiment.

FIG. 17 shows an example of data that would be labeled as ‘none’ for identifying an intermittent pattern in oxygen saturation recordings, in accordance with a representative embodiment.

FIG. 18 shows an example of data that would be labeled as ‘mild’ for identifying an intermittent pattern in oxygen saturation recordings, in accordance with a representative embodiment.

FIG. 19 shows an example of data that would be labeled as ‘severe’ for identifying an intermittent pattern in oxygen saturation recordings, in accordance with a representative embodiment.

FIG. 20 illustrates a technique for using a recurrence-based analysis and a machine-learning technique, in accordance with a representative embodiment.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/of” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “about,” “approximately,” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” and the like, are words of convenience and are not to be construed as limiting terms unless specifically stated to the contrary.

In general, the devices, systems, and methods disclosed herein generally relate to monitoring for, and detecting, a condition related to fluctuations in arterial oxygen saturation (SaO₂) in hemodialysis patients and the like, such as intradialytic sleep apnea and the like. The present teachings may further include devices, systems, and methods for providing a notification and/or an intervention when such a condition is detected. In certain aspects, a blood monitor, which may be disposed in-line within the extracorporeal circuit for a hemodialysis procedure or the like, may sense an attribute of a patient's blood that yields one or more insights regarding the patient's oxygen saturation levels, such as measurements of the patient's oxygen saturation levels during one or more sleep episodes that occur during the hemodialysis procedure. This attribute and/or other data may then be analyzed to determine whether the patient has one or more medical conditions based on hypoxemia (or another oxygen event of interest) experienced during the treatment. The present teachings may include one or more specific techniques for analyzing the patient's oxygen saturation levels—(1) a technique involving a recurrence-based metric and/or a complexity-based metric, and/or (2) a technique involving the use of a machine-learning algorithm trained to identify a presence of an intermittent pattern that would be formed in a plot of oxygen saturation levels experienced by the patient during a hemodialysis procedure. Each of these techniques is described herein, and it will be generally understood that, unless explicitly stated to the contrary or otherwise clear from the context, features described relative to one technique may also or instead be used relative to the other technique, and vice-versa.

It will be understood that an “intermittent pattern” as described herein shall generally include any noteworthy alternation (e.g., irregular alternation) of cyclic dynamics (e.g., between periodic and/or different forms of chaotic dynamics) unless explicitly stated to the contrary or otherwise clear from the context. And, in the context of a preferred embodiment of the present teachings, an intermittent pattern may generally refer to any irregular alternation (and/or any alternation of interest) in SaO₂ levels of a patient over time—where the intermittent pattern would be formed in a plot of the SaO₂ levels over time. Such an intermittent pattern (especially when including relatively high-frequency oscillations) can be an indicator of a patient experiencing and/or having one or more of the following medical conditions, which are provided by way of example and not of limitation: sleep apnea syndrome (SAS), chronic obstructive pulmonary disease, a stroke and/or a proclivity for strokes, hypopnea, hypoxemia, obstructive sleep apnea-hypopnea syndrome (OSAHS), and the like. It will further be understood that an intermittent pattern as described herein may include, in some aspects of the present teachings, a particular pattern such as a sawtooth pattern and the like. Therefore, in some aspects of the present teachings, an intermittent pattern may be characterized by abnormally high frequency and/or high amplitude fluctuations in the SaO₂ levels from a patient undergoing a hemodialysis treatment and the like. In this manner, the present teachings may include techniques to analyze and/or detect abnormal patterns in oximetry data obtained during a hemodialysis procedure or the like.

The present teachings may thus be used to identify an “oxygen-related event” experienced by a patient undergoing a procedure such as a treatment employing extracorporeal circulation, e.g., a hemodialysis treatment. It will be understood that such an oxygen-related event may include any oxygen-related event of interest, which may include, without limitation, one or more of the following: hypoxemia, hypoxia, hypopnea, dyspnea, hyperventilation, hyperpnea, tachypnea, bradypnea, hypoventilation, hypercapnia, nocturnal hypoventilation, sleep apnea syndrome (SAS), chronic obstructive pulmonary disease, a stroke, obstructive sleep apnea-hypopnea syndrome (OSAHS), and the like. Thus, it will be understood that an oxygen-related event may include any condition (including conditions not mentioned above or herein) that could cause high frequency and/or high amplitude fluctuations to a patient's oxygen saturation levels.

Recurrent-Based Time-Series Analysis

In certain aspects, as described above, a blood monitor may sense an attribute of a patient's blood that yields one or more insights regarding the patient's oxygen saturation levels, such as measurements of the patient's oxygen saturation levels during one or more sleep episodes that occur during a hemodialysis procedure or any procedure/treatment employing extracorporeal circulation. This attribute and/or other data may be analyzed using a recurrent-based time-series analysis and/or any measure of time-series complexity to characterize the desaturation in SaO₂ (and, in some instances, arousal from sleep). To this end, one or more of the following metrics may be used: (i) the probability of recurrence for any given state (which may be otherwise referred to herein as a recurrent rate, or RR), (ii) systemic predictability (which may be otherwise referred to herein as determinism, or DET), (iii) rate of occurrence of laminar states (which may be otherwise referred to herein as LAM), (iv) the optimal recurrence threshold as determined by the maximum change in the system determinism value (which may be otherwise referred to herein as ϵₒₚₜᵢₘₐₗ), and (v) permutation entropy to quantify complexity. That is, these quantities may be used to detect episodes of SAS in patients while undergoing hemodialysis treatment. This is because DET, LAM, RR, ϵₒₚₜᵢₘₐₗ, and entropy may contain characteristic properties that can be used to detect the onset of intradialytic sleep apnea-hypopnea syndrome, which may be characterized by an intermittent pattern (e.g., a sawtooth-like and/or other high-frequency oscillatory pattern) in SaO₂. The predictive power of these features and their relationships to clinical outcomes may be useful to the healthcare industry.

Therefore, the present teachings generally include systems and techniques for analyzing oxygen saturation levels sensed during a hemodialysis treatment (or other treatment that includes an extracorporeal circuit) for a patient to determine whether the patient has one or more medical conditions based on an oxygen-related event experienced during the treatment. To this end, the present teachings may include performing a time-series rolling window analysis on a plurality of oxygen saturation levels for the patient including calculating recurrence based metrics, such as, but not limited to: (i) the probability of recurrence for one or more of the plurality of oxygen saturation levels (referred to in the specialized literature as recurrence rate); (ii) the predictability for one or more of the plurality of oxygen saturation levels (referred to in the specialized literature as determinism); (iii) the occurrence of laminar states (referred to as laminarity); (iv) the optimal recurrence threshold, calculated according to the prescription described herein; and (v) calculating complexity metrics, such as, but not limited to, permutation entropy. Moreover, one or more of the aforementioned time series analyses may be used to detect sleep apnea syndrome, e.g., when the oxygen saturation levels are sensed during a sleep event while the patient is undergoing the hemodialysis treatment or the like.

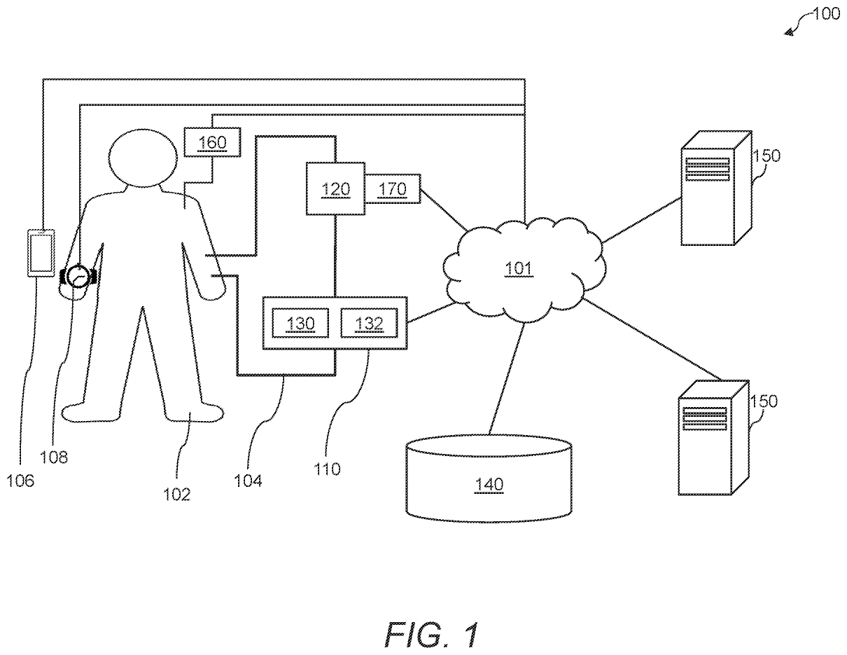

FIG. 1 is a system for monitoring oxygen saturation in hemodialysis patients, in accordance with a representative embodiment. In general, the system 100 may include a patient 102 (e.g., a hemodialysis patient), an extracorporeal circuit 104, a computing device 106, a patient monitor 108, a dialysis machine 110, a blood monitor 120, a processor 130 and a memory 132, a database 140, one or more external resources 150, a data network 101, an intervention mechanism 160, and one or more communications interfaces 170 connecting one or more participants of the system 100 over the data network 101. The system 100 may be configured to monitor for, and detect, a condition related to fluctuations in arterial oxygen saturation in hemodialysis patients, such as intradialytic sleep apnea, as described herein.

The patient 102 may include a kidney patient undergoing a dialysis treatment (which may also be referred to herein as a dialysis procedure). Thus, the patient 102 may include a human that has kidney disease (e.g., acute, or chronic kidney disease), or that has experienced a form of kidney failure, thus needing dialysis treatments. The patient 102 may also or instead include a human that otherwise has an extracorporeal circuit 104 associated therewith in which a blood monitor 120 such as those described herein may be situated. The patient may alternatively include a non-human.

The extracorporeal circuit 104 may include one or more fluid lines and/or connectors for connecting the blood side of the dialysis machine 110 to the vascular system of the patient 102. In general, the extracorporeal circuit 104 may provide a fluid path for drawing blood from the patient 102 through one or more of the dialysis machine 110 and the blood monitor 120, and in a preferred embodiment, back into the vascular system of the patient 102 thereby providing processed blood to the patient 102. The extracorporeal circuit 104 may thus include tubing such as intravenous (IV) tubing made from any suitable material, including without limitation, one or more of polypropylene, nylon, dynaflex, and the like. The fluid lines and/or connectors, or generally the extracorporeal circuit 104, may also or instead include devices or components used to gain access to the blood of the patient 102 for hemodialysis, including without limitation, one or more of an IV catheter, a synthetic graft, and the like.

The extracorporeal circuit 104—or the system 100 more generally—may further include one or more other components such as a pump, a switch (e.g., for adjusting pressure and/or flow settings), a driver, an energy source, and so on. For example, the extracorporeal circuit 104—or the system 100 more generally—may include one or more of a sensor, a pressure monitor (e.g., an arterial pressure monitor, a venous pressure monitor, and the like), an air trap, an air detector, a connector, a valve, a peristaltic pump (e.g., a roller pump), a syringe pump, a centrifugal pump, a heparin pump, a saline drip (or other drip, or pharmaceutical solution), a reservoir, a heater, a controller, a resistive element, a reducer, and the like. For example, in an aspect, a pressure differential for ultrafiltration may be established by adding resistance (through the use of a hemodialysis component including a resistive element) to the extracorporeal circuit 104 (e.g., on a blood outlet on the blood side of the dialysis machine 110). This may be accomplished, e.g., by partially closing a valve on the extracorporeal circuit 104. One or more of these other components may also or instead be included on another participant in the system 100 such as the dialysis machine 110.

The dialysis machine 110 may be any as known in the art, e.g., a standard “off-the-shelf” machine or dialyzer. For example, the dialysis machine 110 may include one or more of a hollow-fiber dialyzer and a plate dialyzer. In general, the dialysis machine 110 may include a semipermeable membrane for the diffusion of one or more solutes therethrough. The dialysis machine 110 may also or instead include one or more other components typically found on such machines such as a sensor, a pump, pressure control mechanisms, a controller, a display, alert/notification interfaces, and so on. In some implementations, the dialysis machine 110 includes a processor 130 and a memory 132 as described herein, although it will be understood that the processor 130 and memory 132 may also or instead be included on one or more other components in the system 100. It will be further understood that the dialysis machine 110 may include another medical mechanism in addition to, or instead of, a traditional dialysis machine 110.

The blood monitor 120 may be structurally configured to sense an attribute of blood of the patient 102 within a portion of the extracorporeal circuit 104 during a hemodialysis procedure over one or more time periods. In this manner, the blood monitor 120 may include one or more sensors. Further, in some aspects, the blood monitor 120 may be the component in the system 100 (or one of a plurality of components) that analyzes the attribute of blood of the patient 102 to provide a plurality of oxygen saturation levels for the patient 102 over one or more time periods. The attribute sensed by the blood monitor 120 may include one or more of hemoglobin, oxygen saturation itself or otherwise an oxygen level, hematocrit, blood volume, and the like. In this manner, in some aspects, the blood monitor 120 may include a Crit-Line® IV monitor (where Crit-Line® is a registered trademark of Fresenius Medical Care) that measures hematocrit, percent change in blood volume, and oxygen saturation in substantially real-time (where “in substantially real-time” may be defined herein as a time period that is perceived to be instantaneous to a human user) for application in the treatment of dialysis patients with the intended purpose of providing a more effective treatment for both the dialysis patient and the dialysis technician. The blood monitor 120 may also or instead include one or more of the devices described in U.S. Pat. No. 9,173,988, which is hereby incorporated by reference in its entirety.

The patient monitor 108 may include a device configured to sense and/or measure a physiological attribute of the patient 102. In this manner, the patient monitor 108 may include one or more sensors. For example, the patient monitor 108 may include a wearable physiological monitoring device. In general, the patient monitor 108 may include a device that can sense, for example, one or more of temperature, heart rate, respiration, blood volume, blood pressure, oxygen saturation levels, alertness, activity, movement, sound, light, and so on. Other physiologic monitoring systems are also or instead possible for use as the patient monitor 108 in the system 100. The patient monitor 108 may also or instead include a component configured to detect when the patient 102 is sleeping, and/or a sleep state of the patient 102. In this manner, the patient monitor 108 may include a camera or other imagining device, or the like.

The intervention mechanism 160 will be understood to include any device, component, user, or technician/clinician (e.g., a human), and the like that is capable of providing an action, intervention, adjustment, alert, and so on for the patient 102 in the system 100. By way of example, the intervention mechanism 160 may include a controller capable of adjusting one or more components of the system 100 such as the dialysis machine 110. The intervention mechanism 160 may also or instead include a device (or human/animal) that is capable of or programmed to awaken the patient 102 from a sleep state, e.g., in response to identification of sleep apnea events during a dialysis treatment. The intervention mechanism 160 may also or instead include an oxygen supply, e.g., for providing supplemental oxygen to the patient 102. The intervention mechanism 160 may also or instead include a supply of medicine for administering to the patient 102. In use cases where the intervention mechanism 160 includes a human, the human may prescribe medication, make a referral (e.g., for polysomnography or the like), and/or perform any other task typically performed by a dialysis technician and/or medical professional. The intervention mechanism 160 may be implemented for use based on substantially real-time information and identification, and/or based on aggregated information over past treatments of the patient 102 or someone similarly situated to the patient 102.

In embodiments where the intervention mechanism 160—or another component of the system 100—includes a controller, such a controller may be configured to start, stop, and/or adjust a component of the system 100 such as the dialysis machine 110 or a subcomponent thereof, such as a pump, a driver, an energy source, and so on. The controller may also or instead be configured to lock a function of, or access to, a component of the system 100, or the system 100 generally. Such control from the controller may be based on signals received from a sensor in the system 100, and/or based on information, instructions, data, and the like received from another component in the system 100, or instructions received from a user or otherwise. In general, a controller in the system 100 may be electrically coupled in a communicating relationship—e.g., an electronic communication—with any of the components of the system 100. In general, the controller in the system 100 may be operable to control the components of the system 100, and may include any combination of software and/or processing circuitry suitable for controlling the various components of the system 100 described herein including without limitation one or more processors 130, microprocessors, microcontrollers, application-specific integrated circuits, programmable gate arrays, and any other digital and/or analog components, as well as combinations of the foregoing, along with inputs and outputs for transceiving control signals, drive signals, power signals, sensor signals, and the like. In certain implementations, the controller in the system 100 may include the processor 130 or other processing circuitry with sufficient computational power to provide related functions such as executing an operating system, providing a graphical user interface (e.g., to a display coupled to a control panel or another component of the system 100), set and provide rules and instructions for operation of a component of the system 100, convert sensed information into instructions, notifications, and the like, and operate a web server or otherwise host remote operators and/or activity through one or more communications interfaces. In certain implementations, the controller in the system 100 may include a printed circuit board, an Arduino controller or similar, a Raspberry Pi controller or the like, a prototyping board, or other computer-related components. A controller in the system 100 may be a local controller disposed on a component of the system 100, or a remote controller otherwise in communication with the system 100 and its components. For example, one or more of a controller and a user interface in communication with the controller may be found on an external resource 150 or a computing device 106 in communication with the system 100 over the data network 101.

The processor 130 may include an onboard processor for a component of the system 100, such as the dialysis machine 110 as shown in the figure. The processor 130 may also or instead be disposed on another device or participant in the system, such as an external resource 150 that is connected to the system 100 or one or more of its components through a data network 101, e.g., using a communications interface 170, which may include a Wi-Fi transmitter and receiver. The processor 130 may perform calculations, e.g., for adjusting a component of the system 100. The processor 130 may also or instead be the component in the system 100 that performs some or all of the analyses discussed herein. The processor 130 may be any as described herein or otherwise known in the art. The processor 130 may be included on the dialysis machine 110 or a controller thereof, or it may be separate from the dialysis machine 110, e.g., it may be included on a computing device 106 or an external resource 150 in communication with a component of the system 100. In an implementation, the processor 130 is included on, or is in communication with, a server that hosts an application, computer model, algorithm, computer program, and the like, for operating and controlling the system 100.

The memory 132 may be any as described herein or otherwise known in the art. The memory 132 may contain computer code and may store data such as sequences of operation, sequences for notifications and alerts, historical data of the system 100, security data, health data, and so on. The memory 132 may contain computer-executable code stored thereon that provides instructions for the processor 130 for implementation by a component of the system 100. The memory 132 may include a non-transitory computer-readable medium. Although shown as included on the dialysis machine 110, similar to the processor 130 described above, the memory 132 may be separate from the dialysis machine 110, e.g., it may be included on a computing device 106 or an external resource 150 in communication with a component of the system 100.

The computing device 106 may include any such device as known in the art, including without limitation a smartphone, a desktop computer, a laptop computer, a network computer, a tablet, a mobile device, a portable digital assistant, a cellular phone, a portable media, or entertainment device, and so on. The computing device 106 may provide a user interface for access to data and analysis by a user, and/or to control the operation of a component of the system 100. The user interface may be maintained by a locally-executing application on the computing device 106, or the user interface may be remotely served and presented on the computing device 106, e.g., from a remote server or the like.

The data network 101 may be any network(s) or internetwork(s) suitable for communicating data and information among participants in the system 100. This may include public networks such as the Internet, private networks, telecommunications networks such as the Public Switched Telephone Network or cellular networks using third-generation (e.g., 3G or IMT-2000), fourth-generation (e.g., LTE (E-UTRA) or WiMAX-Advanced (IEEE 802.16m)), fifth-generation (e.g., 5G), and/or other technologies, as well as any of a variety of corporate area or local area networks and other switches, routers, hubs, gateways, and the like that might be used to carry data among participants in the system 100. This may also include local or short-range communications networks suitable, e.g., for coupling the blood monitor 120 to an external resource 150 and/or database 140, or otherwise communicating with local or remote resources.

The communications interface 170 may be configured to couple one or more participants of the system 100 in a communicating relationship, e.g., over the data network 101. Thus, one or more of the participants in the system 100 may include a communications interface 170. In this manner, communication over the data network 101, or other communication between components of the devices or systems described herein, may be provided via one or more communications interfaces 170. The communications interface 170 may include, e.g., a Wi-Fi receiver and transmitter to allow the logic calculations to be performed on a separate device. This may include connections to smartphone applications and the like. More generally, the communications interface 170 may be suited such that any of the components of the system 100 can communicate with one another. Thus, such a communications interface 170 may be present on one or more of the components of the system 100. The communications interface 170 may include, or be connected in a communicating relationship with, a network interface or the like. The communications interface 170 may include any combination of hardware and software suitable for coupling the components of the system 100 to a remote device (e.g., an external resource 150 such as a remote computer or the like) in a communicating relationship through a data network 101. By way of example and not limitation, this may include electronics for a wired or wireless Ethernet connection or any other short or long-range wireless networking components or the like. This may include hardware for short-range data communications such as Bluetooth or an infrared transceiver, which may be used to couple into a local area network or the like that is in turn coupled to a data network 101 such as the internet. This may also or instead include hardware/software for a WiMAX connection or a cellular network connection (using, e.g., CDMA, GSM, LTE, or any other suitable protocol or combination of protocols). Additionally, a component may be configured to control participation by the components of the system 100 in any network to which such a communications interface 170 is connected, such as by autonomously connecting to the data network 101 to retrieve status updates and the like.

The database 140 may be any as known in the art, and may be local or remote to other components in the system 100. The database 140 may be configured to store data created in the system 100, e.g., data obtained during dialysis treatments and observations related thereto. Thus, in an aspect, the database 140 may include historic data regarding the patient 102 and/or other patients.

One or more of the external resources 150 may include a remote server, which itself may include data storage, a network interface, and/or other processing circuitry. The external resource 150 may process data from the system 100 and perform any of the analyses described herein, and/or may host a user interface for remote access to data and the like, e.g., from the computing device 106. Thus, one or more of the external resources 150 may include a remote server that may include a web server or similar front end that facilitates web-based access by a component of the system 100 to the capabilities of the remote server or other components of the system 100.

The external resources 150 may more generally include any resources that can be usefully employed in the devices, systems, and methods as described herein. For example, these external resources 150 may include without limitation other data networks, human actors (e.g., programmers, researchers, annotators, editors, analysts, and so forth), sensors (e.g., audio, or visual sensors), data mining tools, computational tools, data monitoring tools, algorithms, mathematical models, and so forth. The external resources 150 may also or instead include any other software or hardware resources that may be usefully employed in the networked applications as contemplated herein. For example, the external resources 150 may include certificate servers or other security resources for third-party verification of identity, encryption, or decryption of data, and so forth. In another aspect, the external resources 150 may include a desktop computer or the like co-located (e.g., on the same local area network with, or directly coupled to through a serial or USB cable) with another component of the system 100. In this case, the external resources 150 may provide supplemental functions for components of the system 100. Thus, while depicted as a separate network entity, it will be readily appreciated that the external resources 150 (e.g., a web server) may also or instead be logically and/or physically associated with one of the other devices described herein, and may, for example, include or provide a user interface for web access to a remote server or a database 140 in a manner that permits user interaction through the data network 101, e.g., from the computing device 106.

The external resources 150 may also or instead be in the form of other hardware. In certain implementations, this other hardware may include a camera, other sensor or monitor, or the like. The camera may allow a user to view the system 100 in real-time, or to view recorded information, where such information may be stored on the memory 132 or database 140. This can enable a user to gauge the efficacy of the system 100. The other hardware may also or instead include input devices such as a keyboard, a touchpad, a computer mouse, a switch, a dial, a button, and the like, as well as output devices such as a display, a speaker, other audio transducers, light-emitting diodes or other lighting or display components, and the like. Other hardware of system 100 may also or instead include a variety of cable connections and/or hardware adapters for connecting to, e.g., external computers, external hardware, external instrumentation or data acquisition systems, and the like.

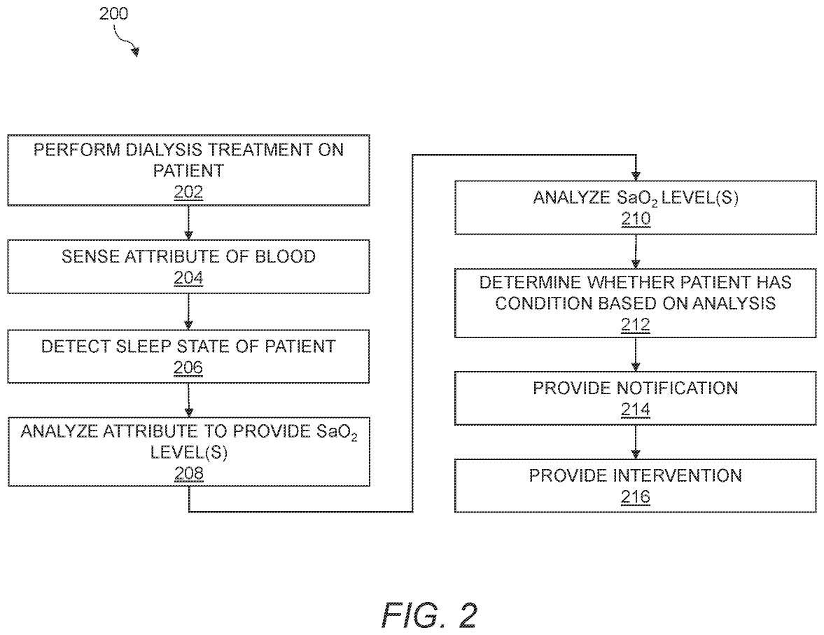

It will be understood that one or more of the steps related to any of the techniques described herein, or sub-steps, calculations, functions, and the like related thereto, can be performed locally, remotely, or some combination of these. Thus, the one or more of the steps of the method 200 described below with reference to FIG. 2 may be performed locally on a component (e.g., the computing device 106, the dialysis machine 110, the blood monitor 120, the processor 130, the intervention mechanism 160, and so on), remotely on a server or other external resource 150, on an intermediate device such as a local computer used by a user to access the external resources 150, or any combination of these. For example, one or more steps of a technique for detecting a condition related to fluctuations in arterial oxygen saturation may wholly or partially be performed locally, such as by training a machine learning model to detect deviations from a baseline for arterial oxygen saturation, and then pruning or otherwise optimizing the machine learning model for deployment on a local component. Also, or instead, one or more steps of a technique for detecting a condition related to fluctuations in arterial oxygen saturation may wholly or partially be performed remotely on one or more of the external resources 150. Thus, for example, data from the blood monitor 120 or the like may be continuously or periodically transmitted to one or more of the external resources 150, which may monitor received data to monitor for deviations from a baseline for arterial oxygen saturation. Other combinations are also or instead possible.

FIG. 2 is a flow chart of a method for monitoring SaO₂ in hemodialysis patients, in accordance with a representative embodiment. The method 200 may be performed, for example, using one or more of the components of the system 100 described above with reference to FIG. 1. In one use case, the method 200 may be used for the detection of intradialytic sleep apnea in hemodialysis patients, and for one or more of a notification and intervention regarding the same, although it will be understood that the method 200 may be used for the monitoring, detection, and subsequent notification and/or intervention of other conditions in addition to or instead of intradialytic sleep apnea.

As shown in step 202, the method 200 may include performing a dialysis treatment on a patient, or another type of treatment employing extracorporeal circulation. As stated above, this may entail the use of a system 100 similar to that described above with reference to FIG. 1.

Turning back to FIG. 2, as shown in step 204, the method 200 may include sensing an attribute of blood of the patient within a portion of an extracorporeal circuit during a hemodialysis procedure. In certain implementations, the attribute includes hemoglobin—and, in this manner, in certain implementations, oxygen saturation is derived from the sensed and/or measured hemoglobin. Additionally, or alternatively, the sensed attribute itself may be oxygen saturation. In some aspects, the method 200 further includes sensing one or more second attributes of blood of the patient within the portion of the extracorporeal circuit during the hemodialysis procedure, in addition to the attribute(s) discussed above. These second attributes may include one or more of hematocrit, blood volume, and the like. Thus, the method 200 may involve sensing a plurality of attributes of the patient's blood, or stated more generally, the method 200 may include sensing a plurality of physiological attributes regarding the patient. It will be understood that these physiological attributes may include attributes regarding the patient's blood and/or other physiological attributes that can be sensed and monitored independently from monitoring within the portion of the extracorporeal circuit that features the patient's blood.

As discussed herein, in certain implementations, the sensing of an attribute of blood of the patient within a portion of an extracorporeal circuit may involve the use of a blood monitor such as an IV monitor—e.g., a Crit-Line® IV monitor (where Crit-Line® is a registered trademark of Fresenius Medical Care) that measures hematocrit, percent change in blood volume, and oxygen saturation in substantially real-time. In certain aspects, the attribute is sensed at a frequency of 1 Hertz (Hz). However, other frequencies are possible, such as any frequency within a range of frequencies from about 0.1 Hz to about 1 Hz, or greater.

The sensing of an attribute of blood of the patient may be conducted over a first period of time, which can include one or more sleep events for the patient. Stated otherwise, the first time period may include one or more episodes of sleep for the patient. In this manner, as shown in step 206, the method 200 may include detecting a sleep state of the patient. By way of example, the sleep state can include a binary state regarding whether or not the patient is asleep, and/or more granular information regarding sleep such as the actual stages of sleep of the patient, e.g., Stage 1, Stage 2, Stage 3, and/or REM sleep. To this end, the method 200 may include monitoring the patient to determine whether the patient is sleeping and/or to determine the sleep state of the patient. Similarly, the method 200 may also or instead include monitoring the patient to determine when the patient is awakened from sleep. In this manner, the method 200 may be used to identify specific sleep episodes experienced by the patient, where each episode has a discernable beginning and ending. These specific sleep episodes may represent salient time periods to analyze the attribute of the patient's blood as sensed by a blood monitor or the like. Thus, the first time period discussed in the method 200 may include one or more such sleep episodes. The first time period may also or instead include a pre-established time period of interest.

Such monitoring of one or more sleep states of the patient may include a visual monitoring of the patient, such as by a video camera, other optical sensors (or other sensors), a human user, combinations thereof, and the like. Such monitoring may also or instead include a monitoring of sound—and, to this end, in certain aspects, the method 200 may include monitoring snoring of the patient. Such monitoring may also or instead include a monitoring of motion of the patient. Such monitoring may also or instead include a monitoring of physiological attributes of the patient that can be used to deduce a sleep state of the patient. To this end, monitoring the patient may be conducted at least in part by a wearable physiological monitor, where such monitors typically include functionality to sense one or more of motion, breathing, heart rate, temperature, and the like.

As shown in step 208, the method 200 may include analyzing the attribute to provide a plurality of oxygen saturation levels for the patient over the first time period. It will be understood that this step 208 may be conducted by a blood monitor itself, and/or this step 208 may be unnecessary where the sensing directly provides oxygen saturation levels as the sensed attributes. For example, certain blood monitors sense and measure hemoglobin, which in turn can yield a value for oxygen saturation derived therefrom.

As shown in step 210, the method 200 may include analyzing the plurality of oxygen saturation levels. At least part of the analysis may be conducted to determine whether one or more of the plurality of oxygen saturation levels is less than a predetermined threshold level by a predetermined amount. It will be understood that the predetermined threshold level can be set by a technician and/or medical professional, and could include any number of levels based on particular circumstances relevant to the patient. By way of example, a predetermined threshold level may be a low end of a typical, normal arterial oxygen range of about 75 to about 100 millimeters of mercury (mm Hg) and/or expressed as percentages such as about 95 to about 100 percent, although other levels are possible. Also, or instead, a predetermined threshold level may be an average of the oxygen saturation for the patient at a given time, e.g., at the beginning of a hemodialysis treatment (e.g., over the first 3 minutes or so). In this manner, the predetermined threshold level may be customized to the patient. Similarly, the predetermined amount below the predetermined threshold level can be set by a technician and/or medical professional, and could include any number of amounts based on particular circumstances relevant to the patient. By way of example, the predetermined amount may be about 3% below the predetermined threshold level. The analysis of the plurality of oxygen saturation levels, at least in part, may also or instead include the application of one or more of a recurrence-based metric and a complexity-based metric to identify a possible medical condition of the patient, as explained in more detail below.

The method 200 may include conducting a time-series analysis on the plurality of oxygen saturation levels for the patient over at least the first time period. The time-series analysis may include calculating one or more recurrence-based metrics. More specifically, the time-series analysis may include calculating recurrence-based quantification analysis such as, but not limited to: (i) probability of recurrence for one or more of the plurality of oxygen saturation levels, (ii) predictability for one or more of the plurality of oxygen saturation levels, (iii) rate of occurrence of laminar states, and/or (iv) optimal recurrence threshold. The time-series analysis may also or instead include (v) calculating a complexity metric, such as, but not limited to, permutation entropy. Each of the time series analyses (i)-(v) may be collectively used to detect sleep apnea syndrome, and/or other conditions of interest. And, in particular, in certain aspects, calculating the predictability, calculating the optimal recurrence threshold, and calculating permutation entropy are analyzed at an onset of a sleep period for detecting sleep apnea syndrome, and/or other conditions of interest. These specific analyses—as well as support for metrics used in such analyses—are discussed in more detail below and in the example study disclosed herein.

Further Detail on Analysis and Support for Techniques Used Herein

Rolling Analysis of Time Series

A rolling analysis of a time series may be used to assess its stability and stationarity over time. Given a time series with N points: (t)={\vec{x}₁, x₂, . . . , x_{N}}, and a function ƒ(\vec{x}) that extracts a single metric from a time series, \vec{x}_{i→j} may denote the section of the time series from tᵢ to tⱼ. The rolling analysis of the time series with window size W and window step S may generate a new time series for the metric, given by:

{ƒ(\vec{x}_{1→W}),ƒ(\vec{x}_{S→S+W}), . . . ,ƒ(\vec{x}_{N-W→N})}.

Permutation Entropy

The permutation entropy (see, e.g., Christoph Bandt and Bernd Pompe, “Permutation Entropy: A Natural Complexity Measure for Time Series,” Phys. Rev. Lett. 88, 174102—Published 11 Apr. 2002, doi: 10.1103/PhysRevLett.88.174102, which is hereby incorporated by reference in its entirety) can be a robust metric for analyzing time series complexity. It may require the specification of a single parameter, (D), called order, which specifies the length (in number of points) of sections of the time series. For use in the present teachings, the value D=10 may be adopted, however, it will be understood that any other value larger than or equal to 3 can be used. Thus, it will be understood that the determination of the value D=10 is somewhat arbitrary—i.e., where 10 was used in the example study, but this could be left free as a parameter, where the value D can be optimized via a meta-optimization scheme or other alternative optimization schemes in order to obtain the best value ranging anywhere from 3 to some fraction of the number of points in the rolling window (i.e., 3≤D<W). For all the possible sections of a given order, the probabilities of occurrence of a given order permutation (πᵢ) may be calculated, and the entropy may be given by:

PE=−Σᵢp(πᵢ)ln p(πᵢ)

Recurrence Quantification Analysis

Recurrence is generally the property of dynamical systems to return to states arbitrarily similar to their initial state, after a sufficiently long time. In time-series analysis, recurrence can be quantified with the recurrence plot (see, e.g., J.-P. Eckmann, S. Oliffson Kamphorst, and D. Ruelle, “Recurrence Plots of Dynamical Systems,” 1987 EPL 4 973, doi.org/10.1209/0295-5075/4/9/004, which is hereby incorporated by reference in its entirety), and can be defined as:

Rᵢⱼ=Θ(ϵ−∥x(tᵢ)−x(tⱼ)∥)

Where E is the recurrence threshold, and any two points x(tᵢ) and x(tⱼ) from the time series may be considered to be recurrent if the distance between them (measured by a given metric ∥⋅∥) is smaller than E.

The recurrence plot Rᵢⱼ may be a binary matrix containing information on the recurrences in the time series. In order to extract this information, several quantifiers can be used such as: recurrence rate, determinism, laminarity, etc. (see, e.g., Norbert Marwan, et al., “Recurrence plots for the analysis of complex systems,” Physics Reports, Volume 438, Issues 5-6, January 2007, Pages 237-329, doi.org/10.1016/j.physrep.2006.11.001, which is hereby incorporated by reference in its entirety). In the present teachings, it will be understood that the focus will generally be on the Determinism (DET), which is a measure of how deterministic the analyzed time series is (periodic time series have determinism close to one, as random ones have determinism close to zero), and it may be defined as:

$$DET ( ϵ ; {ℓ}_{min} ) = \frac{{\sum }_{ℓ = {ℓ}_{min}}^{N} ℓ P ( ℓ , ϵ )}{{\sum }_{ℓ = 1}^{N} ℓ P ( ℓ , ϵ )}$$

Where P( [symbol] ) is a histogram of diagonal lines of length [symbol] in the recurrence plot. A [symbol] -long diagonal line in the recurrence plot may be created whenever the time series has a similar behavior at two distinct times, for at least [symbol] data points. For use in the present teachings, the value [symbol] ₘᵢₙ=10 may be adopted (e.g., when the sampling frequency of the oxygen saturation time series is 1 Hz), however, it will be understood that any other value larger than or equal to 2 can be used. Thus, it will be understood that the determination of the value [symbol] ₘᵢₙ=10 is somewhat arbitrary—i.e., where 10 was used in the example study, but this could be left free as a parameter, where it can range anywhere from 2 to the number of points in the rolling window.

In the present teachings, the value of the Determinism itself may not be of interest, but rather using Determinism to calculate an optimal value for the recurrence threshold E may be desirous (see, e.g., Thiago de Lima Prado, et al., “Optimizing the detection of nonstationary signals by using recurrence analysis,” Chaos 28, 085703 (2018), doi.org/10.1063/1.5022154, which is hereby incorporated by reference in its entirety). The optimal value E may be such that:

$${ϵ}_{optimal} ( {ℓ}_{min} ) = {arg max}_{ϵ} \frac{d DET ( ϵ ; {ℓ}_{min} )}{d ϵ}$$

In this way, the value of ϵₒₚₜᵢₘₐₗ can yield information about the size of the attractor of the system, similarly to its variance, but also can take into consideration the dynamical behavior of the system. Periodic and deterministic systems generally have smaller values of ϵₒₚₜᵢₘₐₗ than stochastic systems, even if they have the same value for the variance.

Other Metrics

Several other metrics may be included in the analysis. For example, the following metrics were also considered for the example study: (1) Kolmogorov Smirnov test between consecutive sub-sections (see, e.g., Kolmogorov-Smirnov Test in The Concise Encyclopedia of Statistics 283-287 (Springer New York, 2008), doi:10.1007/978-0-387-32833-1_214, which is hereby incorporated by reference in its entirety), (2) GARCH model forecasting and coefficients (see, e.g., Dakos, V., et al., “Methods for detecting early warnings of critical transitions in time series illustrated using simulated ecological data,” PLoS One 7 (2012), doi: 10.1371/journal.pone.0041010, which is hereby incorporated by reference in its entirety), (3) critical slowing down analysis, e.g., lag1-autocorrelation and Coefficient of variation (μ/σ) (see, e.g., Dakos, V. & Bascompte, J., “Critical slowing down as early warning for the onset of collapse in mutualistic communities,” Proc. Natl. Acad. Sci. U.S.A. 111, 17546-17551 (2014), doi: 10.1073/pnas.1406326111; and, Rye, C. D. & Jackson, T., “Using critical slowing down indicators to understand economic growth rate variability and secular stagnation,” Sci. Rep. 10, 1-11 (2020), doi: 10.1038/s41598-20-66996-6, which each of the foregoing is hereby incorporated by reference in its entirety), (4) other recurrence quantification analysis (see, e.g., Norbert Marwan, et al., “Recurrence plots for the analysis of complex systems,” Physics Reports, Volume 438, Issues 5-6, January 2007, Pages 237-329, doi.org/10.1016/j.physrep.2006.11.001, which is hereby incorporated by reference in its entirety), e.g., recurrence rate:

$$( i ) RR ( ϵ ) = \frac{1}{{N}^{2}} {\sum }_{i j} {R}_{i j} ,$$

(ii) determinism, (iii) laminarity: which has a similar definition to determinism, but regarding vertical instead of diagonal lines in the RP, (iv) KS-like distance between consecutive histograms of diagonal and vertical lines (i.e., P( [symbol] ) and P(v)), (v) orthogonal variations of the lag 1 Poincaré plot (see, e.g., Marcos, J. V., et al., “Regularity analysis of nocturnal oximetry recordings to assist in the diagnosis of sleep apnoea syndrome,” Med. Eng. Phys. 38, 216-224 (2016), doi: 10.1016/j.medengphy.2015.11.10, which is hereby incorporated by reference in its entirety), and (vi) statistical moments such as mean, variance, skewness, and kurtosis. In addition, the recurrence analysis was also performed under different conditions with embedding (see, e.g., Sauer, T., Yorke, J. A. & Casdagli, M., “Embedology,” J. Stat. Phys. 65, 579-616 (1991), doi: 10.1007/BF01053745, which is hereby incorporated by reference in its entirety), where recurrence rates were kept fixed (see, e.g., Webber, C. L. & Zbilut, J. P., “Recurrence quantification analysis of nonlinear dynamical systems,” Tutorials in contemporary nonlinear methods for the Behavioral Sciences 26-94 (2005) available at https://www.nsf.gov/pubs/2005/nsf05057/nmbs/chap2.pdf, which is hereby incorporated by reference in its entirety.), and optimal E was used (see, e.g., Prado, T. D. L., et al., “Optimizing the detection of nonstationary signals by using recurrence analysis,” Chaos 28, (2018), doi: 10.1063/1.5022154, which is hereby incorporated by reference in its entirety.). Considering all of the possible combinations of different conditions and parameters, over 40 different metrics were calculated and applied to the oxygen saturation time series in the example study. However, it will be understood that the two discussed above can be chosen by feature selection as they may be the most explanatory of occurrence of obstructive sleep apnea-hypopnea syndrome (OSAHS) in a dialysis patient population, the detection of which may represent a preferred use case of the present teachings. Thus, specific metrics for the detection of OSAHS are discussed below by a way of example.

Standard Metrics for Oximetry Data

The diagnostics of OSAHS is based on the Apnea Hypopnea Index (AHI), which is the frequency of occurrence of sleep-related obstructive breathing events per hour of sleep, and according to the American Sleep Disorders Association (ASDS), it can be classified as follows (see, e.g., Schlosshan D, Elliott MW, “Sleep 3: Clinical presentation and diagnosis of the obstructive sleep apnoea hypopnoea syndrome,” Thorax, 2004 April, 59(4):347-52, doi: 10.1136/thx.2003.007179, which is hereby incorporated by reference in its entirety): mild is 5-15 events/hour of sleep, moderate is 15-30 events/hour of sleep, and severe is more than 30 events/hour of sleep. Other classifications are also or instead possible.

The ASDS guidelines dictate that the diagnostic should not be based solely on breathing events, but should also take into account other clinical factors. However, identifying the breathing obstruction events may still be a major component of diagnosing OSAHS. The gold standard diagnosis tool is generally thought to be the full polysomnography (PSG), but this is an expensive and time-consuming technique. Many different works have proposed the use of oximetry data as a surrogate, for its low cost and accessibility. Below, the most common metrics developed to assess the occurrence of OSAHS from oximetry data are briefly described for context: (i) oxygen desaturation index (ODI); (ii) cumulative time below threshold; and (iii) delta index.

Oxygen Desaturation Index and Oxygen Desaturation Density

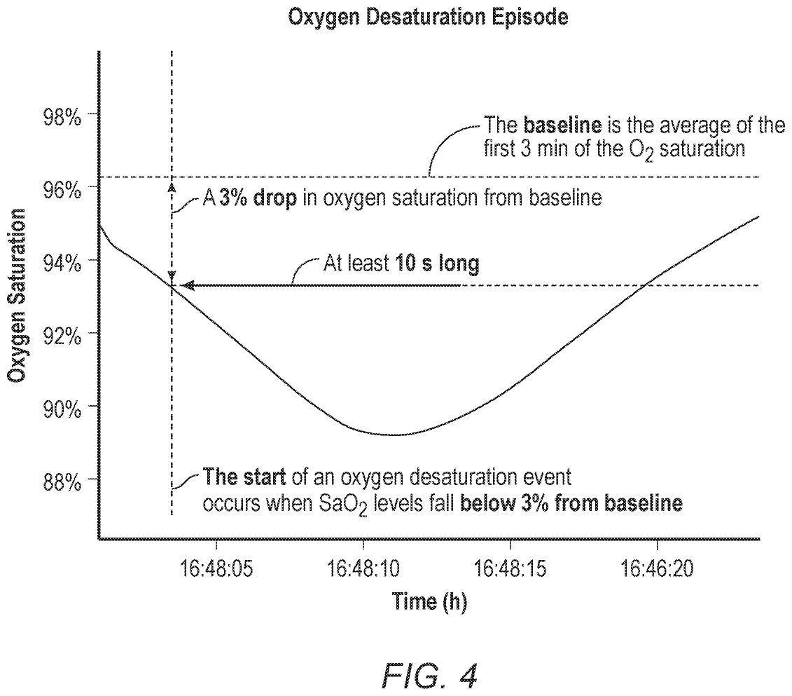

One of the most common uses of oximetry involves the identification of oxygen desaturations, which is most commonly done by searching for sawtooth-like drops in the oxygen saturation time series from a pre-established baseline. There are different ways of determining the baseline, and one of the most common is using the average of the oxygen saturation for a short time (about 3 min) at the beginning of the pertinent time period—e.g., during a hemodialysis treatment. In general, the following procedure may be used for identifying oxygen desaturation, where this procedure was used in the example study disclosed herein: the baseline for each oxygen saturation time series may be determined as the average of the first approximately 3 minutes; a desaturation should drop at least N % from the baseline (e.g., about 3% or about 4%); the desaturation should remain below N % from baseline for a predetermined time period (e.g., for at least about 10 seconds, not exceeding about 60 seconds); and each desaturation time-stamp may be at the instant the oxygen saturation first falls below N % from baseline. FIG. 4 shows an illustrative oxygen desaturation episode represented with details of how the ODI3 was calculated.

A potential problem with this method in identifying breathing obstructions and diagnosing OSAHS may be primarily due to its dependence on a baseline. This is particularly true for patients with other complications, such as chronic kidney disease (CKD) patients undergoing dialysis—in which case, the values of oxygen saturation might be already low at the beginning of a dialysis section, compromising the reliability of the result (see, e.g., Meyring-Wosten, A., et al., “Intradialytic hypoxemia and clinical outcomes in patients on hemodialysis,” Clin. J. Am. Soc. Nephrol. 11, 616-625 (2016), doi: 10.2215/CJN.08510815; and Campos, I., et al., “Intradialytic Hypoxemia in Chronic Hemodialysis Patients,” Blood Purif 41, 177-187 (2016), doi: 10.1159/000441271, which each of the foregoing is hereby incorporated by reference in its entirety).

Once the positions of the oxygen desaturation episodes are properly identified, it may be possible to calculate the oxygen desaturation index or density (ODI or ODD), which has been used as a surrogate for the AHI (see, e.g., Chung, F., et al., “Oxygen desaturation index from nocturnal oximetry: A sensitive and specific tool to detect sleep-disordered breathing in surgical patients,” Anesth. Analg. 114, 993-1000 (2012). doi: 10.1213/ANE.0b013e318248f4f5, which is hereby incorporated by reference in its entirety). The ODIN may represent the number of desaturations at least N % deep below baseline per hours of sleep. It may also be possible to calculate an oxygen desaturation density—at any given point t, the value of the desaturation density may be given by the count of how many desaturations were identified at the time series in a previous time interval (τ), which may be of any length. For example, in the study described herein τ=10 min was used, although any other time span could have been used, where the choice of 10 min was arbitrary and made based on the quality of the observed results. The ODD is actually a time series in which each point ODD(t) counts how many oxygen desaturations episodes took place in the time span of (t−τ, t). In this way, it may be possible to accompany the evolution of the OSAHS in time.

Cumulative Time Below Threshold

Another commonly used metric for identifying hypoxia and OSAHS (and the like) may be the cumulative time below a threshold, which is the ratio of the total time that the oxygen saturation is below a given threshold. It may simply be denoted CT [symbol] , where [symbol] is the threshold—e.g., CT90 represents the cumulative time that SaO₂<90%.

Delta Index

According to Magalang, 2003 (i.e., Magalang, U. J., et al., “Prediction of the Apnea-Hypopnea Index From Overnight Pulse Oximetry,” Chest 124, 1694-1701 (2003), doi: 10.1378/chest.124.5.1694, which is hereby incorporated by reference in its entirety), the Δ index is calculated as “the average of absolute differences of oxygen saturation between successive 12 s intervals (sum of the absolute differences between two successive points, divided by the number of intervals measured).” This can be more formally defined as (see, e.g., Levy, J., et al., “Digital oximetry biomarkers for assessing respiratory function: standards of measurement, physiological interpretation, and clinical use,” npj Digit. Med. 4, (2021), doi: 10.1038/s41746-20-00373-5, which is hereby incorporated by reference in its entirety):

$$Δ = mean [ \frac{1}{τ} {\sum }_{t = 1}^{τ} ‖ Sa {O}_{2} ( t ) - {Sa O}_{2} ( t - 1 ) ‖ ]$$

Where τ is usually a 12-second interval, and the mean is calculated over the entire time series.

More is described below in the example study disclosed herein.

Turning back to FIG. 2 and the method 200 disclosed herein, as shown in step 212, the method 200 may include determining whether the patient has one or more medical conditions based on an oxygen-related event experienced during the hemodialysis procedure from the analysis of the plurality of oxygen saturation levels. By way of example, one or more of the medical conditions may include at least one of sleep apnea syndrome and hypopnea. Thus, and more specifically, the method 200 may include detecting an onset of intradialytic sleep apnea syndrome characterized by an intermittent pattern (e.g., a high-frequency and/or sawtooth-like oscillatory pattern) in oxygen saturation levels based on the time-series analysis.

As shown in step 214, the method 200 may include providing a notification, e.g., a notification regarding an oxygen-related event experienced during the hemodialysis procedure. In some implementations, an oxygen-related event such as hypoxemia may be identified as, for example, when oxygen saturation is less than about 90%, for greater than about 1 min, for at least 5 or more episodes per hour. In certain implementations, the notification is sent to a computing device associated with one or more of the patient, a technician, a medical professional, and the like. Also, or instead, the notification may be provided by at least one of a visual, audio, and/or tactile alert provided by a component associated with the dialysis procedure—e.g., a monitor of the extracorporeal circuit (such as a blood monitor as described herein) and/or a dialysis machine. The notification may be based on information obtained in a current hemodialysis procedure, and/or the notification may be based on information obtained over a plurality of previous hemodialysis procedures.

As shown in step 216, the method 200 may include providing an intervention for the patient. The intervention may include one or more of awakening the patient from sleep, making an adjustment to one or more settings associated with the hemodialysis procedure, prescribing, or conducting polysomnography or another treatment/procedure/study, prescribing or conducting oxygen supplementation, a medication (e.g., administering a medication, prescribing a medication, making an adjustment to existing medication for the patient, and so on), and so forth. The intervention may be based on information obtained in a current hemodialysis procedure, and/or the intervention may be based on information obtained over a plurality of previous hemodialysis procedures.

It will be understood that the method 200 may be performed, in whole or in part, by a computer program product comprising computer-executable code embodied in a non-transitory computer-readable medium that, when executing on one or more computing devices, performs one or more of the steps of the method 200. Further, some or all of the data used in the method 200 may be previously obtained, such that the analysis steps are performed on existing data (e.g., one or more previous samples of information obtained from the patient). Thus, by way of example, obtaining the attribute may be omitted from the method 200 above without departing from the scope of this disclosure.

Example Study

It will be understood that the following description regarding a study conducted using certain aspects of the present teachings is provided by way of example only, and for further understanding of the present teachings.

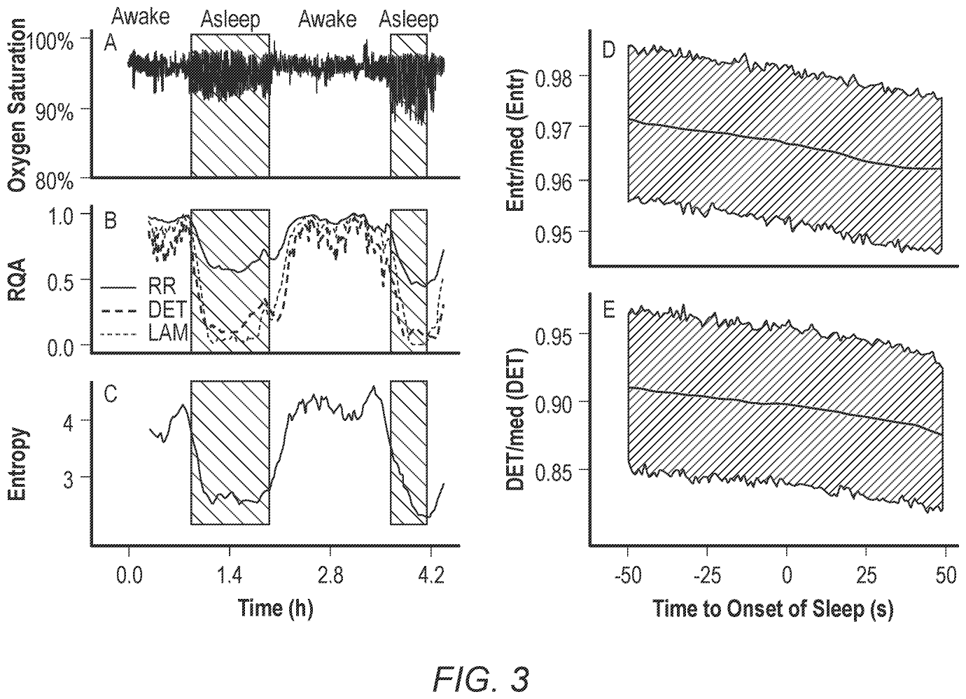

In the example study, 16 hemodialysis (HD) patients with arterio-venous vascular access were studied. Their age was 54±11 years, where 63% were males and 69% were African Americans. Oxygen saturation (SaO₂) was measured during two HD sessions per patient. The mean SaO₂ was 94.3±2.1%. In the example study, distinguishable patterns were observed in which periods where the patient was awake and asleep were differentiated. FIG. 3 shows observations from the example study. In general, FIG. 3 shows a typical SaO₂ annotated with the periods of being awake and asleep (see the shaded area in graph ‘A’ of FIG. 3), during which four of the metrics generally discussed herein decrease during the corresponding period where the patient is asleep (see graphs ‘B’-‘C’ of FIG. 3). Graphs ‘D’-‘E’ of FIG. 3 show the consistency of the metrics in all 16 patients, in which determinism (DET) and entropy were analyzed around the onset of sleep. In the example study, monotonically decreasing trends were observed around the episodes of sleep, suggesting that these results are sensitive to the sleep states, and potentially to an episode of intradialytic sleep apnea syndrome. More specifically, graph ‘A’ of FIG. 3 shows SaO₂ for a representative patient, where the shaded areas annotate the moments of sleep; graph ‘B’ of FIG. 3 shows recurrence quantification analysis (RQA, e.g., RR, DET, and LAM), calculated with a rolling window procedure from the time series of graph ‘A’; graph ‘C’ of FIG. 3 shows permutation entropy calculated with a rolling window procedure from the time series of graph ‘A’; graph ‘D’ of FIG. 3 shows entropy, normalized by its median, and averaged around the onset of sleep for all 16 patients; and graph ‘E’ of FIG. 3 shows determinism, normalized by its median, averaged around the onset of sleep for all 16 patients.

In the example study, oxygen saturation (SaO₂) was measured from the bloodstream by the aforementioned Fresenius Crit-Line® IV monitor with a sampling frequency of 1 Hz, and 32 time series from 16 patients were analyzed, with an average duration of (3.0±0.5) hours. The time series were subjected to the following pre-processing: (i) any value of oxygen saturation smaller than 75% was removed, where the missing values were imputed using a linear interpolation; and (ii) following the procedure described by Schlotthauer, 2014 (Schlotthauer, G., Di Persia, L. E., Larrateguy, L. D. & Milone, D. H., “Screening of obstructive sleep apnea with empirical mode decomposition of pulse oximetry,” Med. Eng. Phys. 36, 1074-1080 (2014), doi: 10.1016/j.medengphy.2014.5.008, which is hereby incorporated by reference in its entirety), the time series were subjected to a low-pass FIR filter with a cutoff frequency of 0.25 Hz (Gustafsson, F., “Determining the initial states in forward-backward filtering,” IEEE Trans. Signal Process. 44, 988-992 (1996), doi: 10.1109/78.492552, which is hereby incorporated by reference in its entirety). Also, a sleep report was generated accompanying the data, in which the time during dialysis that the patient was asleep was reported, with a classification of light sleep and deep sleep. During the example study, a video recording was used to capture periods of wakefulness for treatment sessions.

The analysis techniques used are discussed above in the description of FIG. 2 e.g., using (i) the probability of recurrence for any given state (which may be otherwise referred to herein as a recurrent rate, or RR), (ii) systemic predictability (which may be otherwise referred to herein as determinism, or DET), (iii) occurrence of a laminar state (which may be otherwise referred to herein as LAM), (iv) the recurrence threshold (which may be otherwise referred to herein as ϵₒₚₜᵢₘₐₗ), (v) permutation entropy to quantify complexity; and/or other recurrence-based and/or complexity measures calculated using a rolling window scheme from the SaO₂ time series. As discussed above, these quantities may be used to detect episodes of SAS in patients while undergoing a hemodialysis treatment because DET, LAM, RR, ϵₒₚₜᵢₘₐₗ, and entropy may contain characteristic properties that can be used to detect the onset of intradialytic sleep apnea syndrome, which may be characterized by an intermittent pattern (e.g., a high-frequency and/or sawtooth-like oscillatory pattern) in SaO₂.

The results of this example study can be organized into two sections: retrospective and prospective analysis. The retrospective analysis used the standard oximetry metrics to cluster the patients in two groups: those with OSAHS and those without OSAHS. Based on that clustering, useful metrics to discriminate the patients into these two classes were discovered. The prospective analysis used the selected metrics to predict the value of oxygen desaturation density and classified, in substantially real-time, whether the patient might be experiencing a breathing obstruction due to OSAHS or the like.

Retrospective Analysis

Part 1: Oxygen Desaturation Clustering of Patients

The first step of the analysis may be to determine the patients with OSAHS and check whether the proposed metrics are able to efficiently classify the time series based on this diagnostic. Six standard indexes were calculated for all of the patients in the example study: ODI2, ODI3, ODI4, A, CT90 and CT85, considering the full time series, indiscriminately of the patient being asleep or awake.

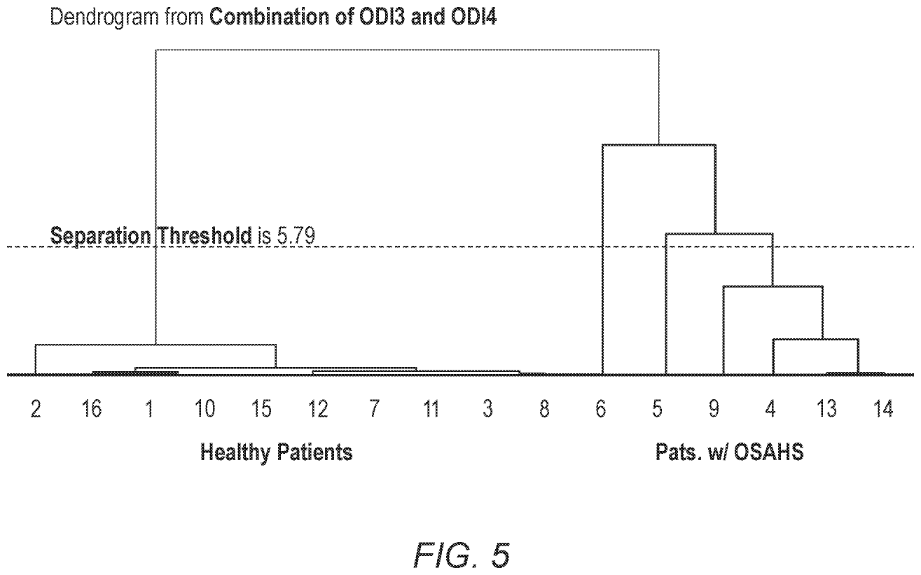

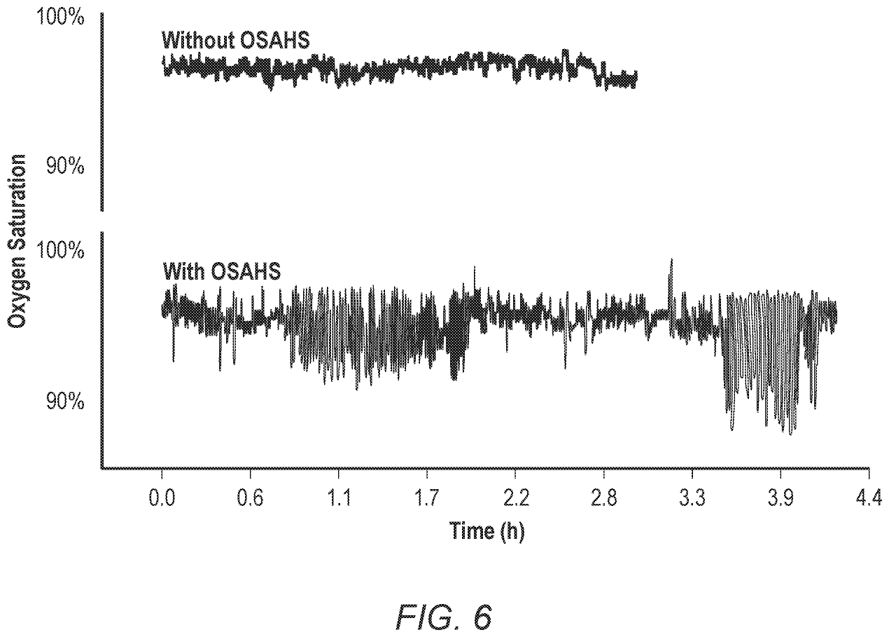

In the example study, a principal component analysis (PCA) was performed, and the first principal component accounts for over 99% of the explained variance. By examining the absolute value of the coefficients of the first principal component, it is possible to determine the most relevant indexes in its composition. As ODI3 and ODI4 are the indexes with the highest coefficients and the most relevant indexes in the composition of the first principal component, and are also the ones more closely related to oxygen desaturations and therefore to breathing obstructions. These are the indexes more commonly used in the literature to identify oxygen desaturations in patients with OSAHS. As a result, these components are the only indexes that were kept in the analysis in the example study. Another index was constructed from a linear combination of ODI3 and ODI4, with the coefficients respecting the relative weights of the PCA. With this other index, a hierarchical clustering was used with weighted pair group method average (see, e.g., Daniel Mullner, “Modern hierarchical, agglomerative clustering algorithms,” (2011), arXiv:1109.2378, which is hereby incorporated by reference in its entirety) to separate the patients in two groups, which are later interpreted as being the groups of patients with OSAHS and without OSAHS. FIG. 5 is a dendrogram with the clustering of the patients in the example study, and FIG. 6 is the time series of two representative patients in the example study described herein, e.g., to illustrate the difference of the oxygen saturation of a patient with OSAHS and a patient without OSAHS. More specifically, FIG. 5 is a dendrogram built from a hierarchical clustering algorithm separating the patients in two classes, with OSAHS and without OSAHS; and FIG. 6 includes two representative time series of patients with and without OSAHS. In FIG. 6, it is possible to see the existing qualitative difference between patients with and without OSAHS. For patients without OSAHS, the oxygen saturation remains constant at relatively high values for the duration of the experiment (except for low amplitude random fluctuations). For patients with OSAHS, it is possible to see sawtooth-like drops of the oxygen saturation (around t≈1.1 h and t≈3.3 h and forward) superimposed to the regular non-OSAHS behavior of the SaO₂. It should be noted that if the value of ODI3 is taken to be a direct replacement for AHI, then the patients selected in the example study would also be the patients selected by using the ASDS criterion for the AHI.

Part 2: OSAHS Classification

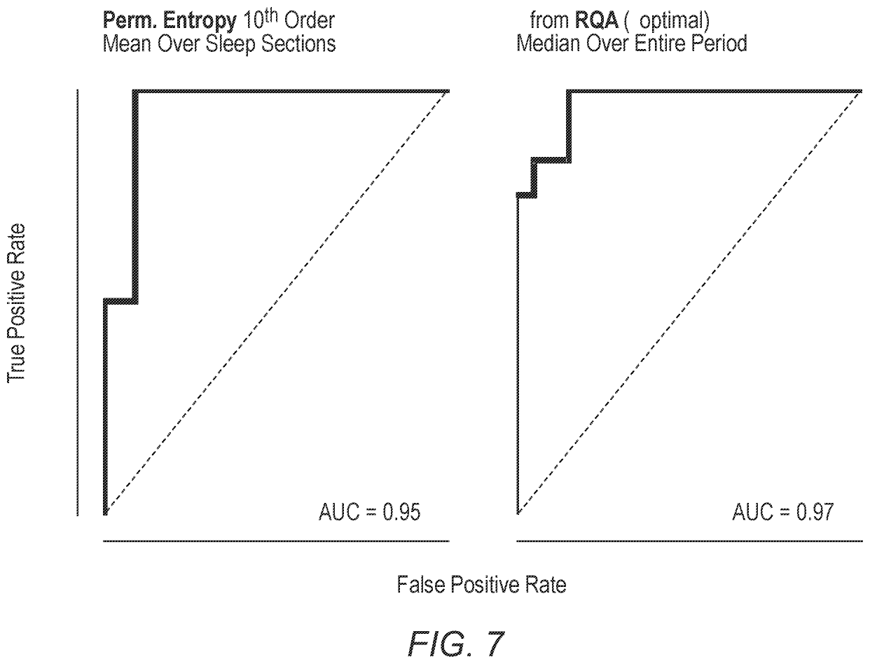

Based on the separation of patients in two classes discussed above in the previous section, the interest in the example study was to determine, from a group of previously selected metrics (see above, i.e., the metrics described in this study were included in this group), which are better at correctly classifying the patients into groups of “with OSAHS” and “without OSAHS.” In order to do that in the example study, the metrics were calculated using the rolling window procedure discussed elsewhere herein. Then, their value was summarized for the entire time series by calculating their mean or median, considering the whole time series or only the reported sleep periods. That is, for each available metric the following four procedures were used to summarize its value for the entire time series: (a) Mean of the metric value for the whole duration of the experiment, (b) Median of the metric value for the whole duration of the experiment, (c) Mean of the metric value only for the periods of reported sleep, and (d) Median of the metric value only for the periods of reported sleep. With the value of the metrics, a Linear Discriminant Analysis (LDA) was performed, which provided a linear combination of the metrics that can better discriminate between the two classes. Based on the absolute value of the coefficients for the LDA, it was possible to select the most discriminatory metrics and evaluate their effectiveness in classifying the patients. FIG. 7 plots the receiver operating characteristic (ROC) curves for the two metrics that most successfully diagnosed the patients in the example study (i.e., it shows ROC curves for the two best-performing metrics for classifying patients based on their OSAHS diagnosis). The selection of the metrics was made based on the value of the area under the curve (AUC) of the ROC. Specifically, the two chosen metrics were: the 10th order permutation entropy and the optimal recurrence threshold for the recurrence plot (ϵₒₚₜᵢₘₐₗ), calculated over sleep periods and the entire dialysis, respectively.

Prospective Analysis

This section regarding the example study includes a discussion regarding the substantially real-time classification based on desaturation density.

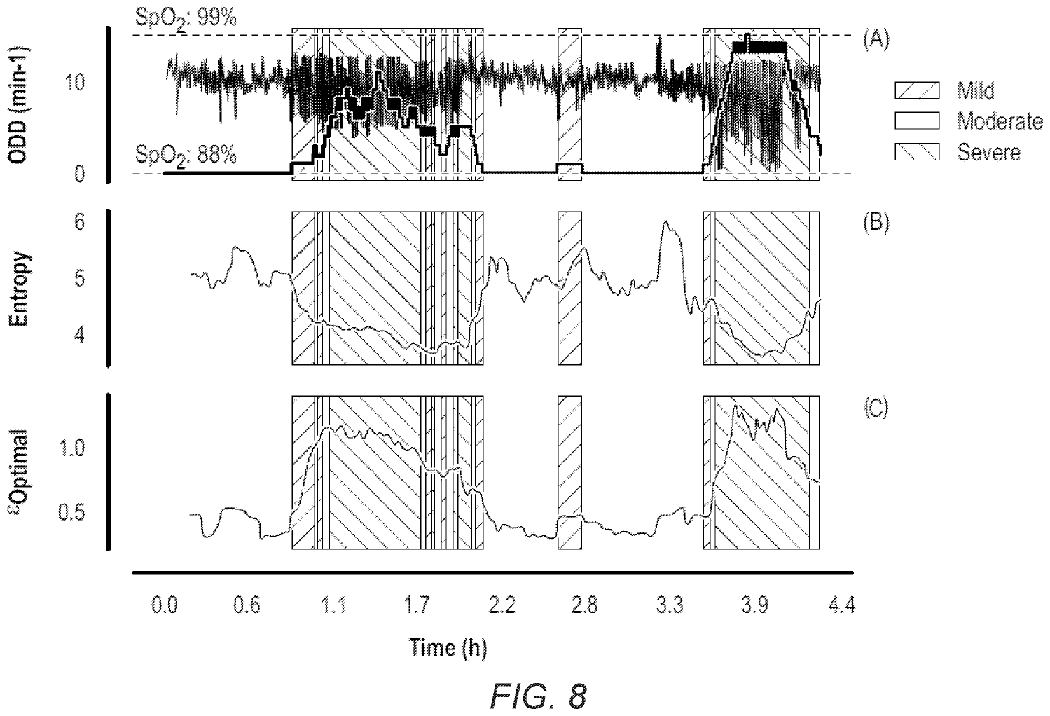

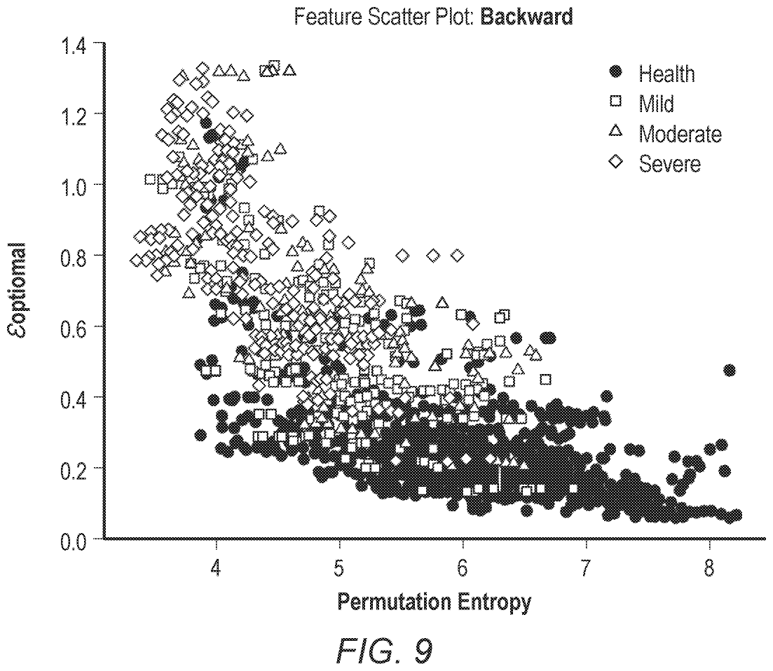

FIG. 8 shows a time series for the oxygen desaturation density (ODD) superimposed to corresponding oxygen saturation (shown in the background), for comparison, as described in the example study discussed herein. That is, the figure shows time series for the oxygen desaturation density (ODD in the relatively dark plotted line), superimposed to the corresponding oxygen saturation for comparison, where the vertical bars mark the region in which the desaturation density reaches levels corresponding to a mild, moderate, and severe OSAHS diagnostic. Specifically, panel (A) in the figure shows an illustrative time series for the 10 min oxygen desaturation density (ODD), superimposed to its corresponding oxygen saturation. It can be seen that the density of desaturation increases whenever there are frequent sawtooth-like drops in oxygen saturation (and/or other intermittent patterns). The vertical bars are to signal the regions in which the oxygen desaturation reaches levels that would correspond to a mild, moderate, and severe OSAHS diagnostic. In panels (B) and (C), the time series for the corresponding metrics that were selected during the retrospective analysis are shown: (B) the permutation entropy; and (C) the ϵₒₚₜᵢₘₐₗ. A visual inspection shows a correlation between the selected metrics and the ODD. To further inspect such correlation, FIG. 9 shows a scatter plot of the two selected features with the corresponding OSAHS intensity color-coded. That is, FIG. 9 is a scatter plot of the metrics for the different intensities of the oxygen desaturation index used in the example study described herein. Because the example study included real-time sleep apnea monitoring, only the periods of the time series with reported sleep were analyzed.

It can be seen in FIG. 9 that patients with and without OSAHS were almost linearly separable in the current feature space. With the exception of the overlap of a few points, the majority of the times in which the patient presented small values of ODD, they also presented large values of entropy and small ϵₒₚₜᵢₘₐₗ; as opposed to larger ODD leading to small entropy and larger ϵₒₚₜᵢₘₐₗ.

This separation of the features with respect to the value of ODD suggested that a real-time classification between patients with and without OSAHS is possible. Considering only reported sleep time, a label was assigned to every patient at every time based on their ODD value. Using the ASDS classification for OSAHS, based on the AHI value, and using the ODD as a direct surrogate for the AHI; for each time instant, one of the following labels was also assigned: “No OSAHS”, “Mild”, “Moderate”, and “Severe.” Then, these labels were translated to two labels “With OSAHS” and “Without OSAHS,” so that the data could be subjected to binary classifiers. The organization of the data in the two labels was made according to three different schemes:
(1) >Mild: “Without OSAHS” was assigned to every instant with the “No OSAHS” label, and “With OSAHS” was assigned to every instant with “Mild”, “Moderate” or “Severe” labels;
(2) >Moderate “Without OSAHS” was assigned to every instant with the “No OSAHS” and “Mild” labels, and “With OSAHS” was assigned to every instant with “Moderate” or “Severe” labels; and
(3) >Severe “Without OSAHS” was assigned to every instant with the “No OSAHS”, “Mild” or “Moderate” labels, and “With OSAHS” was assigned to every instant with “Severe” label.

Based on this new binary classification, after normalization of the features, the data was subjected to a series of different binary classifiers: AdaBoost; Decision Tree; Gaussian Process; Linear Discriminant Analysis (LDA); Linear support vector machines (Linear SVM); Naive Bayes; Nearest Neighbors; Neural Network (Multi-layer perceptron); Quadratic Discriminant Analysis (QDA); Radial basis function support vector machines (RBF SVM); and Random Forest.

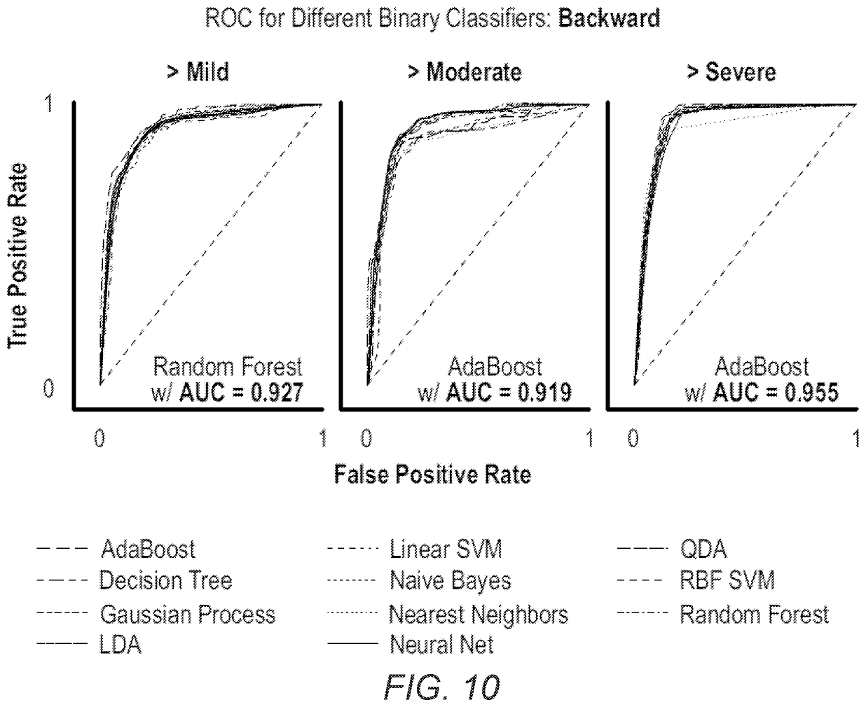

FIG. 10 shows ROC curves for different binary classifiers according to the example study described herein. In FIG. 10, there are the ROC curves for the classifiers, as well as for the three different categorizations of the patients. The area under the curve (AUC) for the best performing classifier is shown on each graph. The performance of the classifiers increases as only the most severe cases are kept in the analysis. Also, although AdaBoost and Random Forest were the best performing classifiers in the example study, all the others presented relatively high AUC suggesting that the metrics proposed are robust features for real-time classification of OSAHS severity.

Therefore, the example study performed a retrospective classification of patients in two classes: with and without OSAHS. The classification was made using features that do not require the calculation of any baseline. Additionally, a prospective analysis was performed in which it was possible to determine, in near real-time, whether or not the patient was experiencing OSAHS related episodes, as would be identified by an increase in the value of the oxygen desaturation density.

It will be understood that the choice of the classification algorithm used in the examples of the present teachings provided herein may be somewhat arbitrary and not determinant to the scope of the present teachings—for example, it will be understood that any robust binary classifier could separate patients regarding the presence of an intermittent pattern (or other abnormal oximetry pattern) given the values of the metrics calculated from the oxygen saturation time series related to the patient. Also, the value of some of the recurrence based metrics and/or complexity based metrics, and/or any possible mathematical combination of these metrics, could be used as potential surrogates on the intensity of an intermittent pattern observed and the like, in which case a simple threshold on such a combination of metrics could be determined to identify the occurrence of an intermittent pattern. Also or instead, any given combination of these metrics could be used as a surrogate for the intensity of an intermittent pattern.

Intermittent Pattern Detection Technique

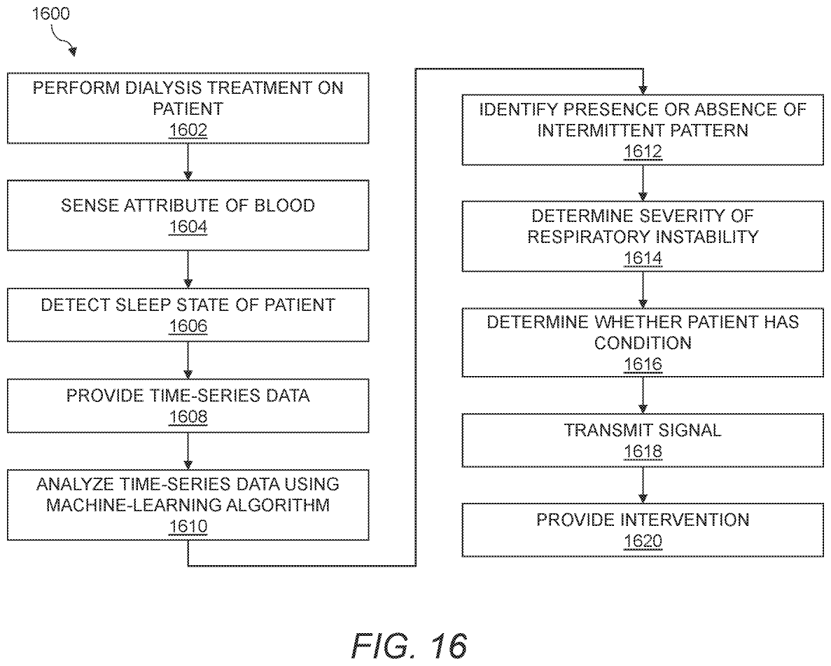

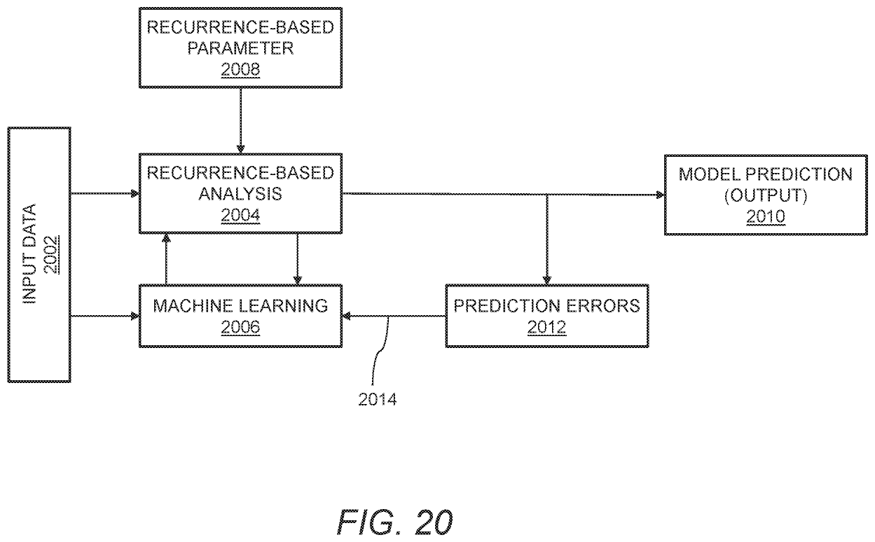

Another technique for detecting intradialytic sleep apnea and other conditions related to fluctuations in arterial oxygen saturation (SaO₂) in hemodialysis patients (and the like) will now be described, where such a technique may include the use of a machine-learning algorithm trained to identify a presence of an intermittent pattern (such as a sawtooth pattern) that would be formed in a plot of oxygen saturation levels experienced by the patient during a hemodialysis procedure or the like. It will be understood that such an intermittent pattern technique may be used as an independent technique to detect an oxygen-related event experienced during a hemodialysis procedure, and/or as a way to complement, supplement, test, train, and the like the technique involving the recurrence-based metric and/or complexity-based metric described above, and vice-versa. By way of example, these techniques may be used together to detect oxygen-related events of interest, and/or the technique involving the recurrence-based metric and/or complexity-based metric described above may be used to train a machine-learning algorithm such as that described below (e.g., the technique involving the recurrence-based metric and/or complexity-based metric described above may be used as a classifier for the machine-learning technique described below), and/or one or more of the techniques may be used as a check/test for another technique, and so on. It will thus be understood that any of the features of the devices, systems, and methods described above with respect to the technique involving the recurrence-based metric and/or complexity-based metric may also or instead be used in the intermittent pattern techniques described herein, and vice-versa. By way of example and not of limitation, the system 100 of FIG. 1 above may be the same or similar to a system used in the intermittent pattern techniques described herein. And, similarly, by way of example and not of limitation, many of the features of the methods of FIGS. 2 and 16 may be used for any of the techniques described herein. Further explanation and examples are provided with reference to FIG. 20 described below.

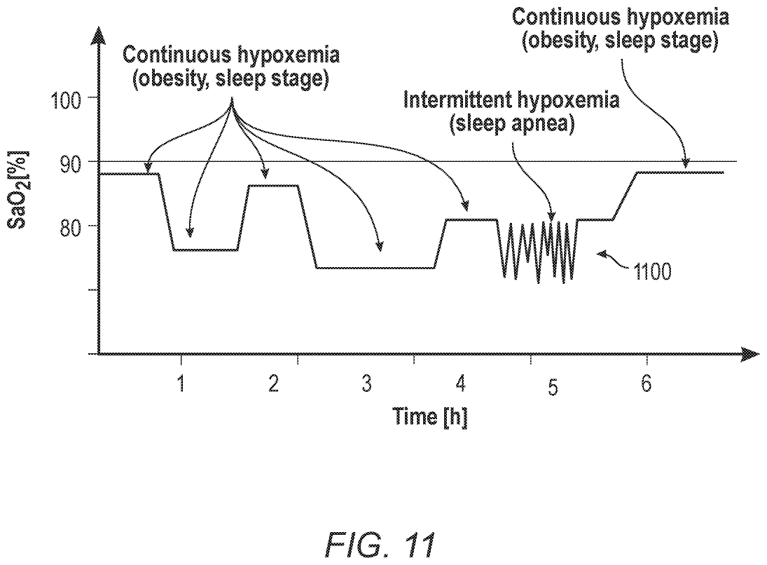

Before describing a technique involving the detection of arterial oxygen saturation episodes during hemodialysis, a brief introduction on intermittent hypoxemia patterns will be described. FIG. 11 is a graph demonstrating continuous and intermittent hypoxemia patterns for context. Specifically, the graph shown in FIG. 11 was retrieved from Dumitrache-Rudjinski S, et al., “The Role of Overnight Pulse-Oximetry in Recognition of Obstructive Sleep Apnea Syndrome in Morbidly Obese and Non-Obese Patients,” Maedica (Bucur), September 2013, 8(3): 237-242. PMCID: PMC3869111 PMID: 24371491, which is incorporated by reference herein.

By way of background, in generally healthy individuals, respiratory control circuits may keep the arterial oxygen saturation (SaO₂) above about 90% (at sea level) at all times. SaO₂ below about 90%, which may also be called hypoxemia, may result from pulmonary pathologies (e.g., COPD) and/or impaired respiratory control. Sleep apnea is a condition where the normal respiration is disrupted by episodes of apnea because of disturbed respiratory control (i.e., central sleep apnea) and/or obstruction of upper airways (i.e., obstructive sleep apnea). And pulse oximetry has been used to demonstrate the characteristic continuous and intermittent patterns (e.g., sawtooth patterns and the like) of SaO₂ desaturations, where the latter may be suggestive of obstructive sleep apnea as demonstrated by the graph in FIG. 11. Thus, FIG. 11 shows an example of an intermittent pattern 1100, where in this example, the intermittent pattern 1100 includes a sawtooth pattern.

In hemodialysis patients, the blood flowing through arterio-venous fistula or arterio-venous graft is essentially arterial, and as such should have an SaO₂ that is equal to or greater than about 95% at sea level. Because hypoxemia is highly prevalent in hemodialysis patients and is associated with increased morbidity, hospitalization, and mortality (see, e.g., Meyring-Wosten A, et al., “Intradialytic Hypoxemia and Clinical Outcomes in Patients on Hemodialysis,” Clin. J. Am. Soc. Nephrol., Apr. 7, 2016, 11(4):616-25, doi: 10.2215/CJN.08510815, which is hereby incorporated by reference), it may be of interest to identify and detect oxygen-related events in hemodialysis patients such as intermittent hypoxemia. To this end, and similar to other techniques described herein, the present teachings may include the sensing of an attribute of blood of a hemodialysis patient within a portion of an extracorporeal circuit. More specifically, the present teachings may involve the use of a blood monitor such as an IV monitor—e.g., a Crit-Line® IV monitor (where Crit-Line® is a registered trademark of Fresenius Medical Care) that measures hematocrit, percent change in blood volume, and oxygen saturation in substantially real-time. And, quasi-continuous measurement of SaO₂ may provide the opportunity to follow the temporal evolution of SaO₂ during hemodialysis.

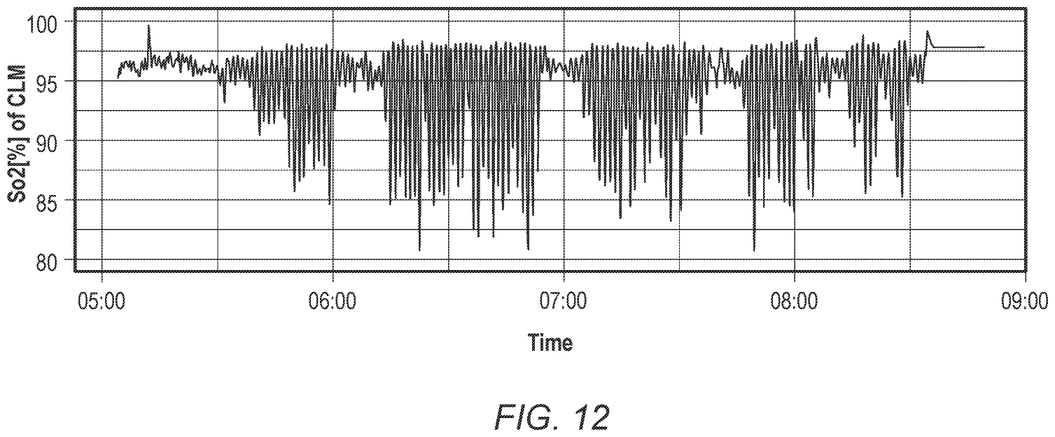

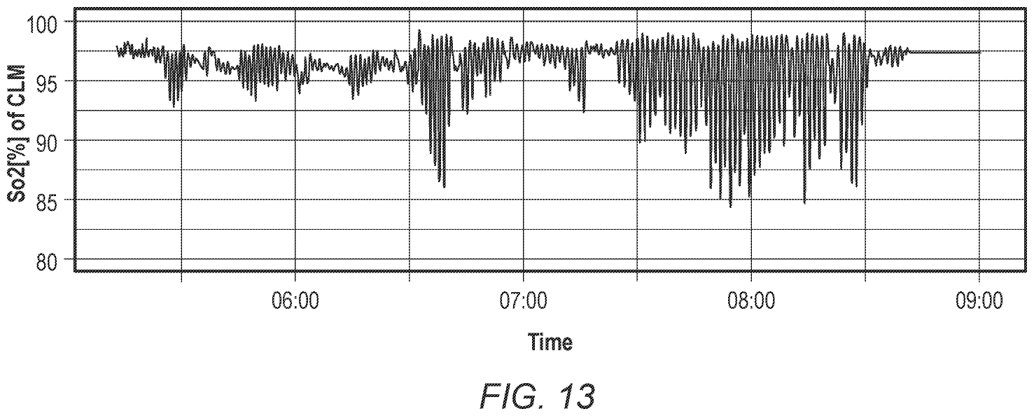

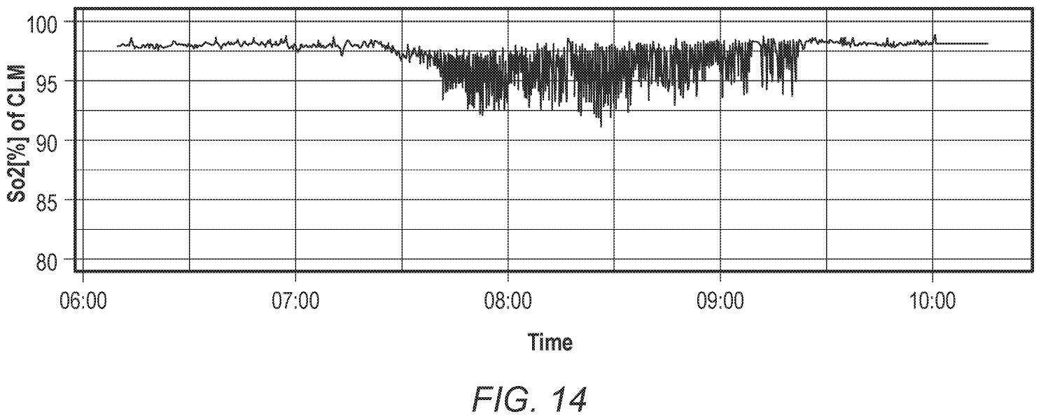

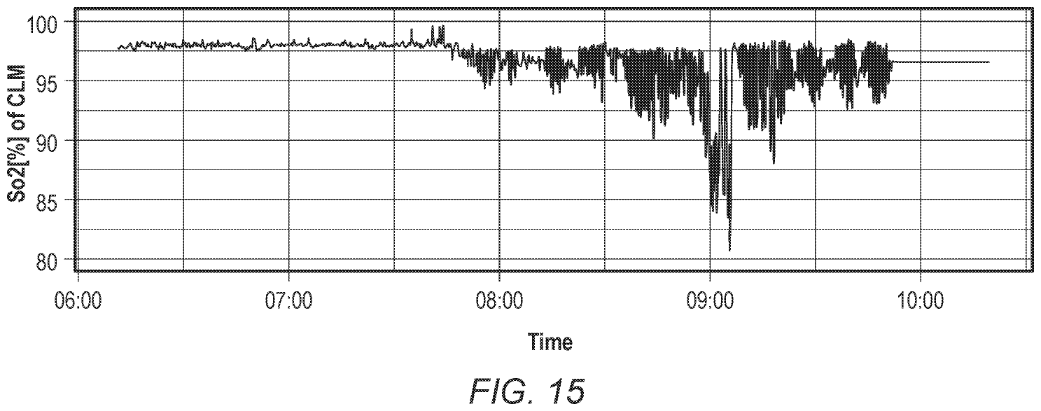

FIGS. 12-15 show examples of intermittent patterns in oxygen saturation levels detected in hemodialysis patients, where, in these examples, the intermittent patterns include sawtooth patterns. Specifically, these examples demonstrate oxygen saturation levels for patients that have been observed to have repetitive episodes of intermittent patterns (e.g., sawtooth patterns) that indicate respiratory instability.

Because it may be impossible (and certainly impractical) to analyze a large number of SaO₂ readings manually, and/or to inspect plots related thereto for intermittent patterns and the like, especially in real time or near real time, the present teachings may include an AI-driven system to automatically identify such intermittent patterns in hemodialysis patients and the like.

FIG. 16 is a flow chart of a method for detecting one or more oxygen-related events experienced during a treatment employing extracorporeal circulation, in accordance with a representative embodiment. For example, the method 1600 may be used for detecting one or more oxygen-related events experienced during a hemodialysis procedure or the like. It will thus be understood that while the method 1600 and certain aspects of the present teachings may be described in the context of a hemodialysis procedure or the like, the method 1600 and certain aspects of the present teachings may also or instead be used in any treatment employing extracorporeal circulation. Similarly, it will be understood that while the method 1600 and certain aspects of the present teachings may be described in the context of identifying and detecting intermittent hypoxemia for the diagnosis of sleep apnea syndrome and the like, the method 1600 and certain aspects of the present teachings may also or instead be used for the identification of any oxygen-related events of interest. For example, while hypoxemia is usually defined by SaO₂<90, using techniques of the present teachings, oxygen events including intermittent behaviors can be seen with and without hypoxemia without departing from the scope of this disclosure. It will also be understood that the method 1600 may be performed, for example, using one or more of the components of the system 100 described above with reference to FIG. 1. By way of example, in one use case, the method 1600 may be used for the detection of intradialytic sleep apnea in hemodialysis patients, and for one or more of a notification and intervention regarding the same, although it will be understood that the method 1600 may be used for the monitoring, detection, and subsequent notification and/or intervention of other conditions in addition to or instead of intradialytic sleep apnea.

As shown in step 1602, the method 1600 may include performing a dialysis treatment on a patient, or another type of treatment employing extracorporeal circulation. As stated above, this may entail the use of a system 100 similar to that described above with reference to FIG. 1.

Turning back to FIG. 16, as shown in step 1604, the method 1600 may include sensing an attribute of blood of a patient within a portion of an extracorporeal circuit during a hemodialysis procedure. In certain implementations, the attribute includes hemoglobin—and, in this manner, in certain implementations, oxygen saturation is derived from the sensed and/or measured hemoglobin. Additionally, or alternatively, the sensed attribute itself may be oxygen saturation. In some aspects, the method 1600 further includes sensing one or more second attributes of blood of the patient within the portion of the extracorporeal circuit during the hemodialysis procedure, in addition to the attribute(s) discussed above. These second attributes may include one or more of hematocrit, blood volume, and the like. Thus, the method 1600 may involve sensing a plurality of attributes of the patient's blood, or stated more generally, the method 1600 may include sensing a plurality of physiological attributes regarding the patient. It will be understood that these physiological attributes may include attributes related to the patient's blood and/or other physiological attributes that can be sensed and monitored independently from monitoring within the portion of the extracorporeal circuit that features the patient's blood.

As discussed herein, in certain implementations, the sensing of an attribute of blood of the patient within a portion of an extracorporeal circuit may involve the use of a blood monitor such as an IV monitor—e.g., a Crit-Line® IV monitor (where Crit-Line® is a registered trademark of Fresenius Medical Care) that measures hematocrit, percent change in blood volume, and oxygen saturation in substantially real-time. In certain aspects, the attribute is sensed at a frequency of 0.1 Hertz (Hz), although it will be understood that other frequencies are possible, such as frequencies up to about 1 Hz or greater.

The sensing of an attribute of blood of the patient may include at least 30 consecutive arterial oxygen saturation recordings. Continuing with this example, these 30 or more consecutive arterial oxygen saturation recordings may be taken about every 10 seconds to represent 1500 measurements in a time-series data for analysis (or, more specifically, readings may be indicative of at least 1500 measurements because each recording, taken every 10 seconds, may represent an average over 10 seconds). That is, in an aspect, for each time series there may be 30 consecutive arterial oxygen saturation recordings (taken at a frequency of every 10 seconds, totaling 5 minutes), where each recording value is an average over 10 seconds, thus representing 1500 measurements. However, it will be understood that these numbers may be somewhat arbitrary, as other numbers and frequencies of arterial oxygen saturation recordings are also or instead possible.

The sensing of an attribute of blood of the patient may be conducted over a first period of time, which can include one or more sleep events for the patient. Stated otherwise, the first time period may include one or more episodes of sleep for the patient. And, thus, sensing the attribute may be conducted during one or more episodes of sleep for the patient. In this manner, as shown in step 1606, the method 1600 may include detecting a sleep state of the patient. By way of example, the sleep state can include a binary state regarding whether or not the patient is asleep, and/or more granular information regarding sleep such as the actual stages of sleep of the patient, e.g., NREM Stage 1, NREM Stage 2, NREM Stage 3, and/or REM sleep. To this end, the method 1600 may include monitoring the patient to determine whether the patient is sleeping and/or to determine the sleep state of the patient. Similarly, the method 1600 may also or instead include monitoring the patient to determine when the patient is awakened from sleep. In this manner, the method 1600 may be used to identify specific sleep episodes experienced by the patient, where each episode has a discernable beginning and ending. These specific sleep episodes may represent salient time periods to analyze the attribute of the patient's blood as sensed by a blood monitor or the like.

Such monitoring of one or more sleep states of the patient may include a visual monitoring of the patient, such as by a video camera, other optical sensors (or other sensors), a human user, combinations thereof, and the like. Such monitoring may also or instead include a monitoring of sound—and, to this end, in certain aspects, the method 1600 may include monitoring snoring of the patient. Such monitoring may also or instead include a monitoring of motion of the patient. Such monitoring may also or instead include a monitoring of physiological attributes of the patient that can be used to deduce a sleep state of the patient. To this end, monitoring the patient may be conducted at least in part by a wearable physiological monitor, where such monitors typically include functionality to sense one or more of motion, breathing, heart rate, temperature, and the like.

As shown in step 1608, the method 1600 may include providing time-series data including a plurality of oxygen saturation levels for the patient. This time-series data may be provided by a blood monitor or the like as described herein, and/or from a device (e.g., a computing device featuring a processor) that is in communication with the blood monitor. For example, a blood monitor or the like may provide “raw” data that is processed to form the time-series data that is provided for analysis as described herein. Thus, the method 1600 may include analyzing the sensed attribute of blood of the patient to provide time-series data including a plurality of oxygen saturation levels for the patient, where this analysis and/or providing of the time-series data may be done by the blood monitor or a similar device, and/or a computing device in communication therewith.

As shown in step 1610, the method 1600 may include analyzing the time-series data using a machine-learning algorithm trained to identify a presence of an intermittent pattern that would be formed in a plot of the plurality of oxygen saturation levels experienced during the hemodialysis procedure. The machine-learning algorithm may be trained using one or more deep learning methods for time series classification. For example, one or more of these deep learning methods may include a one-dimensional convolutional neural network (1D-CNN), which includes state-of-the-art deep learning methods for time series classification. 1D-CNN is one class of deep neural networks, and 1D-CNN includes an input layer (e.g., time series data), multiple hidden layers (e.g., convolution layers, pooling layers, and dense layers), and an output layer (e.g., classification output). As stated throughout this disclosure, the intermittent pattern may include a sawtooth pattern and/or similar patterns of interest.

As shown in step 1612, the method 1600 may include identifying the presence or absence of an intermittent pattern that would be formed in a plot of the plurality of oxygen saturation levels experienced during the hemodialysis procedure. For example, in an aspect, an output of the machine-learning algorithm includes a lack of the presence of such an intermittent pattern. And such a lack of the presence of the intermittent pattern may indicate a lack of intermittent hypoxemia experienced during the hemodialysis procedure. In some instances, the lack of the presence of such an intermittent pattern and/or the indication of the lack of intermittent hypoxemia experienced during the hemodialysis procedure may be a significant result for a particular patient, and thus, the method 1600 may include indicating the lack of intermittent hypoxemia experienced during the hemodialysis procedure, e.g., through the transmission of a signal as described below in step 1618.

As shown in step 1614, the method 1600 may include, when the presence of the intermittent pattern is identified by the machine-learning algorithm, determining a severity of respiratory instability experienced during the hemodialysis procedure. The determination of the severity of respiratory instability experienced during the hemodialysis procedure may be based at least in part on a value of one or more of the plurality of oxygen saturation levels. That is, one or more threshold oxygen saturation levels may be set for a patient, where values for one or more of the plurality of oxygen saturation levels for the patient that fall above or below these thresholds may trigger a certain determination of the severity of respiratory instability experienced during the hemodialysis procedure. By way of example, the determined severity of respiratory instability may include at least one of ‘mild’ and ‘severe.’ And, in certain aspects, the severity of respiratory instability is determined to be severe if a predetermined number of the plurality of oxygen saturation levels are below a certain threshold value such as about 90 percent, about 92 percent, about 95 percent, and so on. The predetermined number of the plurality of oxygen saturation levels that are below a certain threshold value may be a single oxygen saturation level. Stated otherwise, if a single value in the time-series data is below a certain threshold value such as about 90 percent, then the determined severity of respiratory instability may be ‘severe.’ It will be understood that other threshold values, other predetermined number of the plurality of oxygen saturation levels, and/or other severity labels/grades may also or instead be used without departing from the scope of this disclosure.

As shown in step 1616, the method 1600 may include determining whether the patient has one or more medical conditions. This determination may be based on the analysis of the time-series data by the machine-learning algorithm and/or the determined severity of respiratory instability experienced during the hemodialysis procedure. As discussed herein, one or more of the following medical conditions may be determined/identified using the present teachings: sleep apnea syndrome, chronic obstructive pulmonary disease, a stroke, a proclivity for strokes, hypopnea, and the like.

As shown in step 1618, the method 1600 may include transmitting a signal related to an outcome of one or more of the aforementioned analyses. For example, this may include, when the presence of the intermittent pattern is identified by the machine-learning algorithm, transmitting a signal indicating that intermittent hypoxemia was experienced during the hemodialysis procedure. This may instead include transmitting a signal indicating the lack of intermittent hypoxemia experienced during the hemodialysis procedure—e.g., when an output of the machine-learning algorithm includes a lack of the presence of the intermittent pattern, and when the lack of the presence of the intermittent pattern indicates a lack of intermittent hypoxemia experienced during the hemodialysis procedure. Thus, a signal may be transmitted based on the outcome of the aforementioned analyses (e.g., performed at least in part by the machine-learning algorithm) to perform an action (e.g., send a notification, perform an intervention, and the like).

The transmission of the signal in step 1618 may trigger a notification in a system implementing the method 1600. For example, the signal may cause a notification to be sent to a computing device associated with one or more of the patient, a patient caregiver, a technician, a medical professional, and the like—e.g., a mobile phone or the like associated with one or more of the foregoing personnel. Such a notification may be sent during the hemodialysis procedure, e.g., where the notification may be used as a trigger to adjust the hemodialysis procedure. By way of example, a notification may include at least one of a visual, an audio, and a tactile alert provided by a component associated with the hemodialysis procedure—e.g., to inform a person of interest that intermittent hypoxemia or the like was experienced (and/or is currently being experienced) during the hemodialysis procedure. Such a component associated with the hemodialysis procedure may include one or more of a blood monitor, a dialysis machine, and the like. The notification may be based on information obtained in a current hemodialysis procedure. Also or instead, the notification may be based at least in part on information obtained over a plurality of previous hemodialysis procedures—e.g., using historic data for the patient or similar patients. As discussed herein, the signal may trigger an intervention for the patient.

As shown in step 1620, the method 1600 may include providing an intervention for the patient based on the analysis of the time-series data by the machine-learning algorithm and/or the determined severity of respiratory instability experienced during the hemodialysis procedure. By way of example, the intervention may include one or more of awakening the patient from sleep, an adjustment to one or more settings associated with the hemodialysis procedure, polysomnography, oxygen supplementation, a medication, an adjustment to existing medication for the patient, and the like. The intervention may be based on information obtained in a current hemodialysis procedure. Also or instead, the intervention may be based at least in part on information obtained over a plurality of previous hemodialysis procedures—e.g., using historic data for the patient or similar patients.

It will be understood that one or more of the steps of the method 1600, and/or any of the steps or features described herein, may be carried out by a computer program product. For example, in an aspect, a computer program product may include computer-executable code embodied in a non-transitory computer-readable medium that, when executing on one or more computing devices, performs the steps of: sensing an attribute of blood of a patient within a portion of an extracorporeal circuit during a hemodialysis procedure, and analyzing the attribute to provide time-series data including a plurality of oxygen saturation levels for the patient; analyzing the time-series data using a machine-learning algorithm trained to identify a presence of an intermittent pattern that would be formed in a plot of the plurality of oxygen saturation levels experienced during the hemodialysis procedure; when the presence of the intermittent pattern is identified by the machine-learning algorithm, determining a severity of respiratory instability experienced during the hemodialysis procedure based at least in part on a value of one or more of the plurality of oxygen saturation levels; and determining whether the patient has one or more medical conditions based on the analysis of the time-series data by the machine-learning algorithm and/or the determined severity of respiratory instability experienced during the hemodialysis procedure.

Further, and as described herein, although described primarily in the context of a hemodialysis procedure/treatment, the method 1600 may be carried out during any other treatment employing extracorporeal circulation. In this manner, in an aspect, a method for detecting one or more oxygen-related events experienced during a treatment employing extracorporeal circulation may include: sensing an attribute of blood of a patient within a portion of an extracorporeal circuit, and analyzing the attribute to provide time-series data including a plurality of oxygen saturation levels for the patient; analyzing the time-series data using a machine-learning algorithm trained to identify a presence of an intermittent pattern that would be formed in a plot of the plurality of oxygen saturation levels experienced during extracorporeal circulation; when the presence of the intermittent pattern is identified by the machine-learning algorithm, determining a severity of respiratory instability experienced during extracorporeal circulation based at least in part on a value of one or more of the plurality of oxygen saturation levels; and determining whether the patient has one or more medical conditions based on the analysis of the time-series data by the machine-learning algorithm and the determined severity of respiratory instability experienced during extracorporeal circulation.

Further, it will be understood that the method 1600, and/or any of the embodiments of the present teachings discussed herein, may be used in other settings, including those employing extracorporeal circulation as well as those that do not necessarily employ extracorporeal circulation. By way of example and not of limitation, an example use case for the present teachings may include identification of fetal hypoxemia and the like.

It will be further understood that the method 1600 may be carried out in a system such as that shown and described above with reference to FIG. 1. By way of example, such a system may include an extracorporeal circuit connected to a patient for performing a hemodialysis procedure, a dialysis machine within the extracorporeal circuit, a blood monitor within the extracorporeal circuit, and a computing resource configured to receive time-series data including a plurality of oxygen saturation levels for the patient. The computing resource may include computer-executable code embodied in a non-transitory computer-readable medium that, when executing on the computing resource, performs the steps of: analyzing the time-series data using a machine-learning algorithm trained to identify a presence of an intermittent pattern that would be formed in a plot of the plurality of oxygen saturation levels experienced during the hemodialysis procedure; when the presence of the intermittent pattern is identified by the machine-learning algorithm, determining a severity of respiratory instability experienced during the hemodialysis procedure based at least in part on a value of one or more of the plurality of oxygen saturation levels; and determining whether the patient has one or more medical conditions based on the analysis of the time-series data by the machine-learning algorithm and the determined severity of respiratory instability experienced during the hemodialysis procedure. The computing resource may be local or remote, or some combination thereof. For example, the computing resource may be disposed remote from the blood monitor, and may communicate with the blood monitor over a data network.

As discussed above, in certain aspects, a blood monitor such as a Crit-Line® IV monitor (where Crit-Line® is a registered trademark of Fresenius Medical Care) may record oxygen saturation levels every 10 seconds where the data is streamed into a cloud platform in real time; and, for every 30 consecutive oxygen saturation recordings (i.e., a 5 minute interval) during the treatment, these arterial oxygen saturation time series may be analyzed via a machine-learning algorithm to achieve an output as follows: none (indicating no intermittent pattern, e.g., no sawtooth pattern), mild (indicating a mild intermittent pattern, e.g., a mild sawtooth pattern), or severe (indicating a severe intermittent pattern, e.g., a severe sawtooth pattern). The machine-learning algorithm may be trained based on the same type or a similar type of data sets from a plurality of hemodialysis treatments—e.g., 4,000 or more such data sets. That is, data from a plurality of hemodialysis treatments may be used to create labels of ‘none,’ ‘mild,’ and ‘severe.’ The figures below show examples of each of these labels.

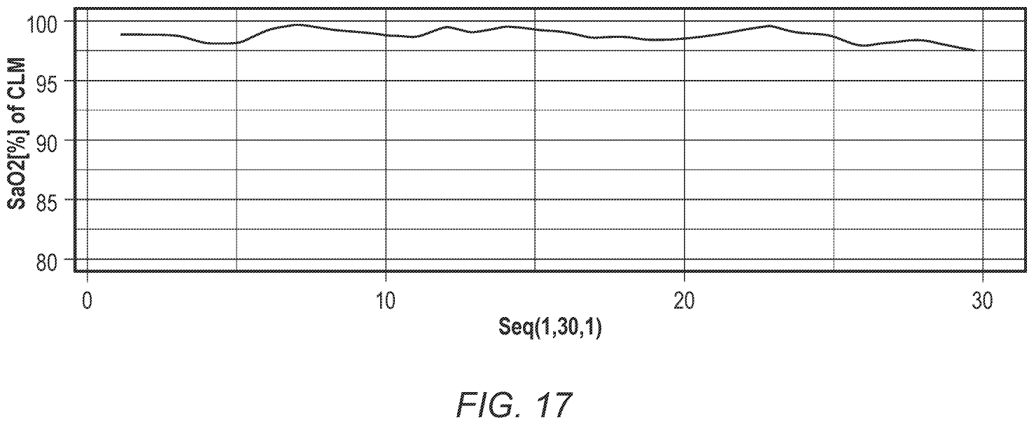

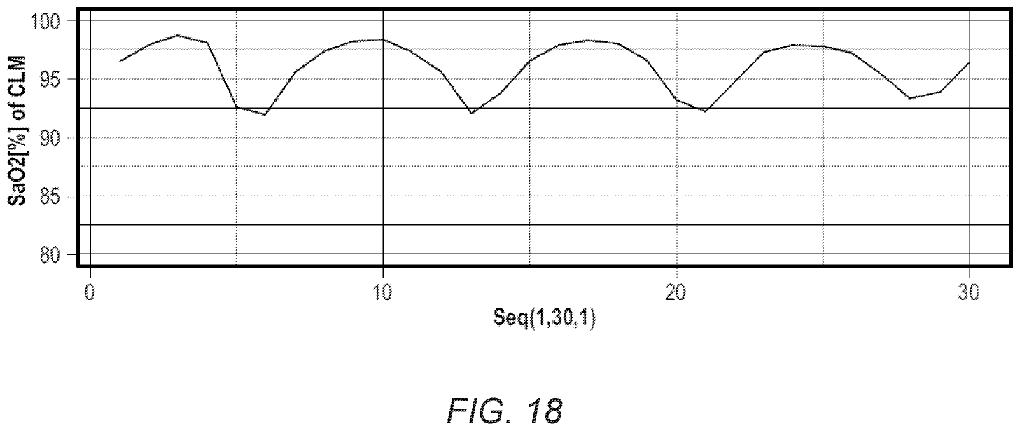

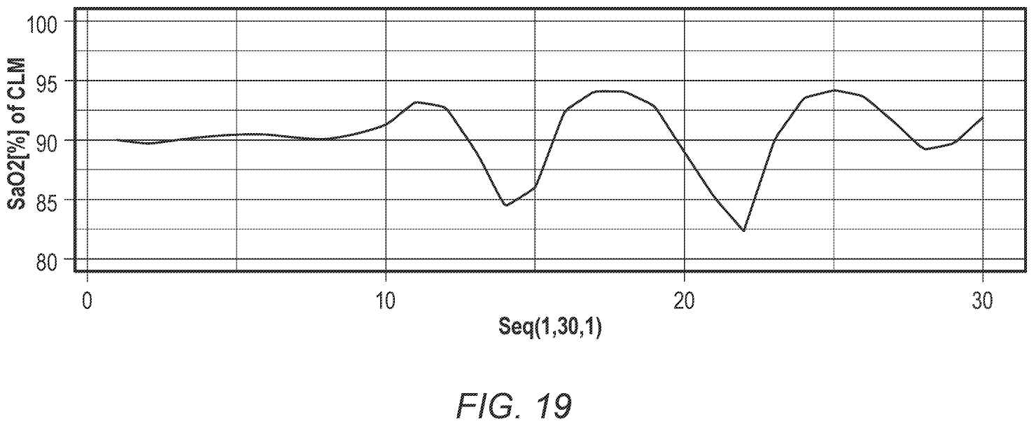

FIG. 17 shows an example of data that would be labeled as ‘none’ for identifying an intermittent pattern (e.g., a sawtooth pattern) in oxygen saturation recordings; FIG. 18 shows an example of data that would be labeled as ‘mild’ for identifying an intermittent pattern (e.g., a sawtooth pattern) in oxygen saturation recordings; and FIG. 19 shows an example of data that would be labeled as ‘severe’ for identifying an intermittent pattern (e.g., a sawtooth pattern) in oxygen saturation recordings. In particular, these figures (and/or their associated labels or grades) may represent outputs of the present teachings, e.g., outputs of a machine-learning algorithm. These figures may instead represent manually labeled data sets that are used to train a machine learning algorithm according to the present teachings. It will be understood that, because there may be a massive difference in the occurrences of these particular labels, certain techniques may be used for training the machine learning algorithm such as those described below.

By way of example, an aspect of the present teachings utilized a total of 4.75 labeled arterial oxygen saturation time series, which included the following distribution for the three categories referenced above: 78.1% none, 10.6% mild, and 11.2% severe. For this example embodiment of the present teachings, for the training data, the resample technique was used in order to achieve a balanced data set. And for the test data, the original distribution was used. Moreover, one-dimensional convolutional neural networks (1D-CNN) were used, which include deep learning methods for time series classification—1D-CNN is one class of deep neural networks, where 1D-CNN includes an input layer (time series data), multiple hidden layers (convolution layers, pooling layers, and dense layers), and an output layer (classification output). With the test data, an accuracy of 93.87% was achieved using the present teachings. For example, 95.8% of ‘none’ time series were classified by the algorithm correctly, 91.2% of ‘mild’ time series were classified by the algorithm correctly, and 82.8% of ‘severe’ time series were classified by the algorithm correctly. Thus, it was demonstrated that generally the predicted outputs were aligned to the actual outputs. And therefore, the algorithm was demonstrated to provide intermittent pattern recognition for an arterial oxygen saturation time series. And, with the current state of connected devices, this intermittent pattern recognition could be performed in real time during a dialysis treatment, providing timely alerts for mild or severe intermittent pattern (e.g., sawtooth pattern) detections. And, as such, this can lead to interventions and/or adjustments in response to an identification of mild or severe intermittent pattern(s) during a dialysis treatment such as one or more of awakening a patient from sleep, polysomnography, oxygen supplementation, adjustments to medication, and the like. And, in some embodiments, an intervention may be based on real time information and identification, and/or from aggregated information over past treatments.

FIG. 20 illustrates a technique for using a recurrence-based analysis and a machine-learning technique, in accordance with a representative embodiment. That is, and as stated above, two of the techniques described herein for detecting oxygen-related events in hemodialysis patients and the like-(i) a technique including the use of a machine-learning algorithm trained to identify a presence of an intermittent pattern (such as a sawtooth pattern), and (ii) a technique utilizing a recurrence-based metric and/or complexity-based metric—may be used together, e.g., as a way to complement, supplement, test, provide inputs, provide labels, and/or train one another. A few of example of combining these techniques are described below, including a technique illustrated in FIG. 20.

As referenced above in this disclosure, a technique using a recurrence-based analysis and a machine-learning technique may include a voting approach. In such a voting approach, both recurrence-based and machine learning methods can be performed independently, where a voting scheme can then be used to select the prediction of the most-accurate technique. Additionally or alternatively, a probability of a prediction may be used—e.g., via Bayesian formalism—to provide the prediction probability given both techniques. Stated otherwise, another example of where the technique involving the recurrence-based metric and/or complexity-based metric described herein and the intermittent pattern technique(s) described herein may be usefully employed together includes running each of these two techniques independently and then performing a voting scheme (e.g., algorithmically) or the like to uncover a result (e.g., in some instances, a more-accurate result rendered than by using one technique alone).

It will be understood that there are multiple ways in which the output of both techniques could be combined, including without limitation: arithmetic mean, geometric mean, median, weighted mean, and the like. Using a voting approach, prior knowledge of the probability of occurrence of an intermittent pattern in the analyzed time series segment can be incorporated (and it will be understood that this probability could be given by any other external analysis, which may not necessarily be dependent on the analysis of the oxygen saturation time series but by any other means of diagnostics used in a clinical context, for example). For example, where w is the prior probability of occurrence of intermittent patterns, and pi represents the probabilities associated with the techniques described in this disclosure (or any other methods that one would like to include in the analysis), one possible way of calculating the combined probability P would be:

$$\hat{p} = \frac{{\sum }_{i = 1}^{N} {w}_{i} {p}_{i} + {w}_{π} π}{{\sum }_{i = 1}^{N} {w}_{i} + {w}_{π}}$$

Where wt corresponds to appropriately chosen weights. Such weights could be chosen, e.g., as the inverse of each technique's error rate (i.e., their precision) in a manner such that better performant methods would have higher weights. The average method described herein is one of many that could be applied.

Another example may include a serial combination of a recurrence-based analysis and a machine-learning technique. That is, in a serial combination, a recurrence-based analysis as described herein can be used to provide labeling for a machine-learning technique described herein. In particular, and by way of example, the optimal recurrent threshold ϵₒₚₜᵢₘₐₗ may behave as a threshold for the existence (or lack thereof) of intermittent dynamics—that is, it can be interpreted as an indicator of intermittent dynamics, or lack thereof. This combination of both techniques can be particularly suited for circumstances that potentially require retraining of the machine learning technique, e.g., when presented with a new data set with slightly different properties, or when there is an interest in analyzing segments of the time series of lengths different from 5 minutes (or another predetermined time period), or when analyzing a time series with a different sampling frequency, and so on.

In general terms, this combination may be organized in the following general steps:
1. calculate ϵₒₚₜᵢₘₐₗ using a rolling window scheme over the time series, with an appropriate length of the rolling window;
2. conduct an exploratory study on a subset of the new data set to determine appropriate thresholds for ϵₒₚₜᵢₘₐₗ, i.e., determine values ϵₘᵢₙ and εₘₐₓ such as: if ϵₒₚₜᵢₘₐₗ<ϵₘᵢₙ then the segment is classified as “without intermittent pattern”, and if ϵₒₚₜᵢₘₐₗ>ϵₘₐₓ then the segment is classified as “with intermittent pattern”;
3. use the established thresholds to label the entire data set;
4. feed the machine learning algorithm with the pairs (time series segments, label) and train the algorithm; and
5. use the trained machine learning algorithm to make predictions on new unobserved data.

Another example may include a sandwich combination of a recurrence-based analysis and a machine-learning technique. In a sandwich combination, when intermediary values for a recurrent threshold (e.g., 0.4<epsilon<1) are present, a machine-learning technique may be used to determine the probability of prediction for the existence (or lack thereof) of intermittent dynamics. Thus, the present teachings may include using the technique involving the recurrence-based metric and/or complexity-based metric described herein—and more specifically, an optimal recurrent threshold thereof—to provide a label for the machine learning method that identifies intermittent patterns. That is, the recurrence-based analysis can provide a “without intermittent pattern” label for values smaller than a threshold ϵₘᵢₙ, and a “with intermittent pattern” label for values of ϵₒₚₜᵢₘₐₗ larger than the threshold ϵₘₐₓ. However, for intermediate values of ϵₒₚₜᵢₘₐₗ multiple approaches can be used to correctly classify the given time series segment. The sandwich combination of both techniques thus may use the machine learning technique in this range of intermediate values of ϵₒₚₜᵢₘₐₗ, providing a probability of observing an intermittent pattern in the oxygen saturation time series segment. By way of example, the recurrence threshold (E, epsilon)<εₘᵢₙ may be used to imply that no intermittent pattern is present, epsilon≥εₘₐₓ may be used to imply the presence of an intermittent pattern, and epsilon between εₘᵢₙ and εₘₐₓ may be used to imply that there is a probability of the presence of an intermittent pattern. Thus, in this manner, the machine learning method described herein may be used to determine an intermittent pattern (e.g., to identify a presence and/or a severity thereof) when epsilon is between εₘᵢₙ and εₘₐₓ. And, in some aspects, if epsilon is outside of these values, computational resources may be preserved by foregoing application of the machine learning technique described herein. Stated otherwise, when the recurrence threshold is greater than or equal to εₘᵢₙ but less than εₘₐₓ, a technique according to the present teachings may include analyzing the plurality of oxygen saturation levels of a patient using a machine-learning algorithm trained to identify a presence of an intermittent pattern that would be formed in a plot of the plurality of oxygen saturation levels, and/or analyzing the plurality of oxygen saturation levels by assigning a probability of occurrence of an intermittent pattern as a function of the recurrence threshold. The values of each of εₘᵢₙ and εₘₐₓ could be one of a plurality of values depending on the sampling frequency of the time series and of parameters specific to the method used. Thus, it will be understood that εₘᵢₙ may be a predetermined minimum value and that εₘₐₓ may be a predetermined maximum value. By way of example, in an aspect, εₘᵢₙ may be about 0.4 and εₘₐₓ may be about 1. However, it will be understood that, under different circumstances, different values could be used that would yield similar results.

Turning to FIG. 20, the present teachings may include an interactive combination of a recurrence-based analysis and a machine-learning technique as described herein. As shown in the figure, input data 2002 (e.g., the time-series data as described herein) may be provided to one or more computing devices associated with one or more of a recurrence-based analysis 2004 and a machine-learning technique 2006. For example, in an aspect, the input data 2002 is supplied to a machine for implementing the recurrence-based analysis 2004, where the recurrence-based analysis 2004 is implemented to predict one or more assigned components of a deep learning method (e.g., a 1D-CNN)—e.g., using a recurrence-based parameter 2008. That is, the recurrence-based analysis 2004 may provide recurrence-based metrics for intermittency conditions to the machine-learning technique 2006. The machine-learning technique 2006 may then use these metrics as labeling to detect refined intermittency criteria, which are iteratively performed while minimizing the prediction errors 2012. The prediction errors 2012 may be used to further refine the characteristics of the machine learning technique 2006 during a learning phase 2014, where, more specifically, the prediction error 2012 may be used to calculate weights and biases (e.g., weights of the convolution, or filters, kernels, other hyperparameters, and so on). And thus, for example, in an aspect, the input data 2002 is supplied to a machine for implementing the machine-learning technique 2006. The machine-learning technique 2006 may be implemented to compute a predicted output (shown as model prediction (output) 2010 in the figure). And, in a learning phase 2014, one or more prediction errors 2012 may be used to calculate proper weights and bases of the deep learning method (e.g., a 1D-CNN).

The above systems, devices, methods, processes, and the like may be realized in hardware, software, or any combination of these suitable for a particular application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. This may also, or instead, include one or more application-specific integrated circuits, programmable gate arrays, programmable array logic components, or any other device or devices that may be configured to process electronic signals. It will further be appreciated that a realization of the processes or devices described above may include computer-executable code created using a structured programming language such as C, an object-oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. In another aspect, the methods may be embodied in systems that perform the steps thereof, and may be distributed across devices in a number of ways. At the same time, processing may be distributed across devices such as the various systems described above, or all of the functionalities may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all of the steps thereof. The code may be stored in a non-transitory fashion in a computer memory, which may be a memory from which the program executes (such as random-access memory associated with a processor), or a storage device such as a disk drive, flash memory, or any other optical, electromagnetic, magnetic, infrared, or other device or combination of devices. In another aspect, any of the systems and methods described above may be embodied in any suitable transmission or propagation medium carrying computer-executable code and/or any inputs or outputs from the same.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings.

Unless the context clearly requires otherwise, throughout the description, the words “comprise,” “comprising,” “include,” “including,” and the like are to be construed in an inclusive sense as opposed to an exclusive or exhaustive sense; that is to say, in a sense of “including, but not limited to.” Additionally, the words “herein,” “hereunder,” “above,” “below,” and words of similar import refer to this application as a whole and not to any particular portions of this application.

It will be appreciated that the devices, systems, and methods described above are set forth by way of example and not of limitation. For example, regarding the methods provided above, absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computing) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A method for detecting an oxygen-related event experienced during a hemodialysis procedure, the method comprising:
sensing an attribute of blood of a patient within a portion of an extracorporeal circuit during a hemodialysis procedure over a first time period, and analyzing the attribute to provide a plurality of oxygen saturation levels for the patient over the first time period;
analyzing the plurality of oxygen saturation levels, wherein the analysis at least in part includes determining whether one or more of the plurality of oxygen saturation levels is less than a predetermined threshold level by a predetermined amount, and application of one or more of a recurrence-based metric and a complexity-based metric to identify a possible medical condition of the patient; and
determining whether the patient has one or more medical conditions based on the oxygen-related event experienced during the hemodialysis procedure based on the analysis of the plurality of oxygen saturation levels.

2. The method of claim 1, wherein the oxygen-related event includes one or more of hypoxemia, apnea, hypopnea, and hypoxia.

3. The method of claim 1, wherein the first time period includes one or more episodes of sleep for the patient, and wherein the one or more medical conditions includes sleep apnea syndrome.

4. The method of claim 1, further comprising conducting a time-series analysis on the plurality of oxygen saturation levels for the patient over at least the first time period, the time-series analysis including calculating one or more recurrence-based metrics including at least one of: (i) calculating a probability of recurrence (recurrence rate) for one or more of the plurality of oxygen saturation levels; (ii) calculating predictability (determinism) for one or more of the plurality of oxygen saturation levels; (iii) identifying a rate of occurrence of one or more laminar states (laminarity); (iv) calculating an optimal recurrence threshold; and (v) calculating a complexity metric.

5. The method of claim 4, wherein each of the time series analyses (i)-(v) are collectively used to detect sleep apnea syndrome.

6. The method of claim 1, further comprising conducting a time-series analysis on the plurality of oxygen saturation levels for the patient over at least the first time period, the time-series analysis including calculating a complexity metric including at least permutation entropy.

7. The method of claim 1, further comprising conducting a time-series analysis on the plurality of oxygen saturation levels for the patient over at least the first time period, and detecting an onset of intradialytic sleep apnea syndrome characterized by one or more intermittent patterns in oxygen saturation levels based on the time-series analysis.

8. The method of claim 1, further comprising monitoring the patient to determine whether the patient is sleeping.

9. The method of claim 1, further comprising providing a notification regarding the oxygen-related event experienced during the hemodialysis procedure.

10. The method of claim 1, further comprising providing an intervention for the patient.

11. The method of claim 10, wherein the intervention includes at least one of: awakening the patient from sleep; an adjustment to one or more settings associated with the hemodialysis procedure; polysomnography; oxygen supplementation; and a medication.

12. The method of claim 10, wherein the intervention is based on information obtained in a current hemodialysis procedure.

13. The method of claim 10, wherein the intervention is based at least in part on information obtained over a plurality of previous hemodialysis procedures.

14. The method of claim 1, wherein the attribute includes hemoglobin.

15. The method of claim 1, wherein the attribute itself is oxygen saturation.

16. The method of claim 1, wherein the attribute is sensed at a frequency of 1 Hertz (Hz).

17. The method of claim 1, wherein the predetermined amount is about 3% below the predetermined threshold level.

18. A system, comprising:
an extracorporeal circuit connected to a patient for performing a hemodialysis procedure;
a dialysis machine within the extracorporeal circuit;
a blood monitor within the extracorporeal circuit; and
a computing resource configured to receive data from the blood monitor related to a plurality of oxygen saturation levels for the patient over a first time period, the computing resource comprising computer-executable code embodied in a non-transitory computer-readable medium that, when executing on the computing resource, performs the steps of: analyzing the plurality of oxygen saturation levels, wherein the analysis at least in part includes one or more of (i) determining whether one or more of the plurality of oxygen saturation levels is less than a predetermined threshold level by a predetermined amount, and (ii) application of one or more of a recurrence-based metric and a complexity-based metric to identify a possible medical condition of the patient; and determining whether the patient has one or more medical conditions based on an oxygen-related event experienced during the hemodialysis procedure from the analysis of the plurality of oxygen saturation levels.

19. The system of claim 18, wherein the computing resource is disposed remote from the blood monitor, and communicates with the blood monitor over a data network.

20. A method for detecting an oxygen-related event experienced during a hemodialysis procedure, the method comprising:
sensing an attribute of blood of a patient within a portion of an extracorporeal circuit during a hemodialysis procedure over a time period, and analyzing the attribute to provide a plurality of oxygen saturation levels for the patient over the time period;
analyzing the plurality of oxygen saturation levels, wherein the analysis at least in part includes application of a recurrence-based metric yielding a recurrence threshold to identify presence of the oxygen-related event;
when the recurrence threshold is less than a predetermined minimum value, εₘᵢₙ, determining a lack of presence of the oxygen-related event;
when the recurrence threshold is greater than or equal to a predetermined maximum value, εₘₐₓ, determining that the oxygen-related event is present; and
when the recurrence threshold is greater than or equal to εₘᵢₙ but less than εₘₐₓ, analyzing the plurality of oxygen saturation levels over the time period using a machine-learning algorithm trained to identify a presence of an intermittent pattern that would be formed in a plot of the plurality of oxygen saturation levels.
